SEQUENCE LISTING (17 sequences)

SEQ ID NO: 1 - DNA, 10 bp, Artificial Sequence
  Chemically synthesized, Tn5 insertional target recognition sequence
  misc_feature (2)..(2): any nucleotide
  misc_feature (8)..(8): any nucleotide
gntywranct

SEQ ID NO: 2 - DNA, 14 bp, Artificial Sequence
  Chemically synthesized, sequence bias
  misc_feature (3)..(3): any nucleotide
  misc_feature (7)..(7): any nucleotide
  misc_feature (13)..(13): any nucleotide
gtnywrnactgtnc

SEQ ID NO: 3 - DNA, 39 bp, Artificial Sequence
  Chemically synthesized, NexA2
agacgtgtgctcttccgatcagatgtgtataagagacag

SEQ ID NO: 4 - DNA, 19 bp, Artificial Sequence
  Chemically synthesized, ME comp
ctgtctcttatacacatct

SEQ ID NO: 5 - DNA, 25 bp, Artificial Sequence
  Chemically synthesized, ExA2.1-N5
  misc_feature (21)..(25): random nucleotide sequence
gacgtgtgctcttccgatctnnnnn

SEQ ID NO: 6 - DNA, 20 bp, Artificial Sequence
  Chemically synthesized, ExA2.1-20
agatcggaagagcacacgtc

SEQ ID NO: 7 - DNA, 63 bp, Artificial Sequence
  Chemically synthesized, ExA1-58-N5
  misc_feature (59)..(63): random nucleotide sequence
aatgatacggcgaccaccgagatctacactctttccctacacgacgctcttccgatctnnnnn

SEQ ID NO: 8 - DNA, 70 bp, Artificial Sequence
  Chemically synthesized, ExA2_iNN
  misc_feature (25)..(36): barcode sequence
caagcagaagacggcatacgagatnnnnnnnnnnnngtgactggagttcagacgtgtgctcttccgatct

SEQ ID NO: 9 - DNA, 33 bp, Artificial Sequence
  Chemically synthesized, ExA2B
gatcggaagagcacacgtctgaactccagtcac

SEQ ID NO: 10 - DNA, 15 bp, Artificial Sequence
  Chemically synthesized, FX-15
ctggagttcagacgt

SEQ ID NO: 11 - DNA, 19 bp, Artificial Sequence
  Chemically synthesized, ME sequence
agatgtgtataagagacag

SEQ ID NO: 12 - DNA, 58 bp, Artificial Sequence
  Chemically synthesized, ExA1-58
aatgatacggcgaccaccgagatctacactctttccctacacgacgctcttccgatct

SEQ ID NO: 13 - DNA, 13 bp, Artificial Sequence
  Chemically synthesized, ExA1-13
gatcggaagagcg

SEQ ID NO: 14 - DNA, 19 bp, Artificial Sequence
  Chemically synthesized, ExA1-SSL_N5
  misc_feature (1)..(5): random nucleotide sequence
nnnnnagatcggaagagcg

SEQ ID NO: 15 - DNA, 18 bp, Artificial Sequence
  Chemically synthesized, P1.3
aatgatacggcgaccacc

SEQ ID NO: 16 - DNA, 69 bp, Artificial Sequence
  Chemically synthesized, NexA2-iNN
  misc_feature (26)..(37): barcode sequence
caagcagaagacggcatacgagatcnnnnnnnnnnnntgactggagttcagacgtgtgctcttccgatc

SEQ ID NO: 17 - DNA, 22 bp, Artificial Sequence
  Chemically synthesized, P2.1
caagcagaagacggcatacgag

(19) United States
(12) Patent Application Publication
(10) Pub. No.: US 2019/0264201 A1
(43) Pub. Date: Aug. 29, 2019

(54) DNA LIBRARY CONSTRUCTION OF IMMOBILIZED CHROMATIN IMMUNOPRECIPITATED DNA

(71) Applicant: The Penn State Research Foundation, University Park, PA (US)

(72) Inventors: Benjamin Franklin Pugh, Boalsburg, PA (US); Matthew John Rossi, State College, PA (US)

(21) Appl. No.: 16/284,001

(22) Filed: Feb. 25, 2019

Related U.S. Application Data
(60) Provisional application No. 62/636,229, filed on Feb. 28, 2018.

(51) Int. Cl. C12N 15/10; C12Q 1/6855; C12Q 1/6869; C12Q 1/6806; C12Q 1/686

(52) U.S. Cl. CPC C12N 15/1093; C12Q 1/6855; C12Q 1/6869; C40B 50/06; C12Q 1/686; C12Q 2600/16; C12N 15/1006; C12Q 1/6806

(57) ABSTRACT

Disclosed herein are compositions and methods for construction of chromatin immunoprecipitation (ChIP) sequencing libraries involving the use of Tn5 tagmentation, splint ligation and/or single-stranded DNA ligation.

### CROSS REFERENCE TO RELATED APPLICATIONS

This application claims priority to U.S. Provisional Application No. 62/636,229, filed Feb. 28, 2018 which is hereby incorporated by reference herein in its entirety.

### STATEMENT OF GOVERNMENT SUPPORT

This invention was made with government support under Grant No. ES013768 and CA168104 awarded by the National Institutes of Health. The government has certain rights in the invention.

### BACKGROUND

Chromatin immunoprecipitation (ChIP) is a long-standing method for detecting protein-DNA interactions in vivo (Solomon and Varshaysky, (1985) Proc Natl Acad Sci USA, 82:6470-6474; Gilmour and Lis, (1984) Proc Natl Acad Sci USA, 81:4275-4279). Formaldehyde is used to covalently trap proteins at their in vivo binding locations. After quenching, chromatin is isolated and fragmented. Next, a protein of interest is immunoprecipitated and its attached DNA identified by either PCR, microarrays (Blat and Kleckner, (1999) Cell, 98:249-259), or deep sequencing (ChIP-seq) (Albert et al., (2007) Nature, 446:572-576; Johnson et al., (2007) Science, 316:1497-1502); listed in order of increased genome coverage and resolution. ChIP-exo was developed as a variation of ChIP-seq to improve sensitivity and increase positional resolution by up to two orders of magnitude. It uses lambda exonuclease to digest sonicated chromatin to the formaldehyde-induced protein-DNA cross-linking point (Rhee and Pugh, (2011) Cell, 147:1408-1419). By providing near base pair (bp) resolution of protein-DNA interactions, structural insights into protein complex organization are gained. The ChIP-exo method was introduced for the SOLiD sequencing platform in 2011 (referred to herein as version 1.0 or ChIP-exo 1.0), followed by an Illumina-based method (referred to herein as version 1.1 or ChIP-exo 1.1) in 2013 (Serandour et al., (2013) Genome Biol, 14:R147; Yen et al., (2013) Cell, 154:1246-1256). A significant drawback of ChIP-exo 1.0 and ChIP-exo 1.1 is their technical complexity compared to the lower resolution ChIP-seq assay. This has limited its broader adoption.

In an effort to simplify ChIP-exo library construction, ChIP-nexus (referred to herein as version 2 or ChIP-exo 2) was developed in 2015 (He et al., (2015) Nature biotechnology, 33:395-401), in which the intermolecular 2^{nd} adapter ligation was replaced by an intramolecular ligation. Despite this improvement, both version 1 and 2 of ChIP-exo remain technically difficult and costly.

Accordingly, there is a need for improved methods that permit rapid, sensitive, and accurate library construction for use with chromatin immunoprecipitation assays. The present invention fulfills this need.

### SUMMARY

In one embodiment, the invention relates to a method for identifying a binding site of a protein of interest, the method comprising the steps of: a) immunoprecipitating the protein of interest bound to a nucleic acid molecule, b) contacting the immunoprecipitated nucleic acid molecule with at least one 5′→3′ exonuclease to generate a single-stranded nucleic acid region on the nucleic acid molecule, c) ligating a first adaptor molecule to the immunoprecipitated nucleic acid molecule while it remains immobilized, d) eluting the nucleic acid molecule, e) ligating a second adaptor molecule to the eluted nucleic acid molecule, 0 amplifying the eluted nucleic acid molecule, and g) sequencing the amplified products.

In one embodiment, step c) comprises ligating the first adaptor molecule by a method selected from the group consisting of tagmentation, 5′ ssDNA ligation, 3′ ssDNA ligation, splint ligation of an adaptor molecule having a 5′ ssDNA overhang, and split ligation of an adaptor molecule having a 3′ ssDNA overhang. In one embodiment, the adaptor molecule comprises a 5′ ssDNA overhang comprising at least 2 random nucleotides at the 5′ end of the 5′ overhang. In one embodiment, the adaptor molecule comprises a 3′ ssDNA overhang comprising at least 2 random nucleotides at the 3′ end of the 3′ overhang.

In one embodiment, step e) comprises ligating the second adaptor molecule by a method selected from the group consisting of tagmentation, 5′ ssDNA ligation, 3′ ssDNA ligation, splint ligation of an adaptor molecule having a 5′ ssDNA overhang, and split ligation of an adaptor molecule having a 3′ ssDNA overhang. In one embodiment, the adaptor molecule comprises a 5′ ssDNA overhang comprising at least 2 random nucleotides at the 5′ end of the 5′ overhang. In one embodiment, the adaptor molecule comprises a 3′ ssDNA overhang comprising at least 2 random nucleotides at the 3′ end of the 3′ overhang.

In one embodiment, the nucleic acid molecule is crosslinked to the protein of interest, and the method further comprises a step of reversing the crosslinks after ligation of a first adaptor molecule.

In one embodiment, the method further comprises a step of end repair prior to exonuclease digestion.

In one embodiment, step c) is performed prior to step b).

In one embodiment, the method further comprises performing A-tailing prior to ligation of a first adaptor molecule.

In one embodiment, the method further comprises a step of phosphorylating a 5′ end of a nucleic acid molecule.

In one embodiment, the step of phosphorylating a 5′ end of a nucleic acid molecule is performed concurrently with step c).

In one embodiment, the method further comprises contacting the nucleic acid molecule with a polymerase to generate a completely dsDNA molecule by filling any ssDNA gaps in the nucleic acid molecule prior to step b).

In one embodiment, the invention relates to a method for identifying a binding site of a protein of interest, the method comprising the steps of: a) immunoprecipitating the protein of interest bound to a nucleic acid molecule, b) ligating a first adaptor molecule to the immunoprecipitated nucleic acid molecule, c) ligating a second adaptor molecule to the immunoprecipitated nucleic acid molecule, d) eluting the nucleic acid molecule, e) amplifying the eluted nucleic acid molecule, and f) sequencing the amplified products.

In one embodiment, step b) is performed concurrently with step c).

In one embodiment, step b) comprises ligating the first adaptor molecule by a method selected from the group consisting of tagmentation, 5′ ssDNA ligation, 3′ ssDNA ligation, splint ligation of an adaptor molecule having a 5′ ssDNA overhang, and split ligation of an adaptor molecule having a 3′ ssDNA overhang. In one embodiment, the adaptor molecule comprises a 5′ ssDNA overhang comprising at least 2 random nucleotides at the 5′ end of the 5′ overhang. In one embodiment, the adaptor molecule comprises a 3′ ssDNA overhang comprising at least 2 random nucleotides at the 3′ end of the 3′ overhang.

In one embodiment, step c) comprises ligating the second adaptor molecule by a method selected from the group consisting of tagmentation, 5′ ssDNA ligation, 3′ ssDNA ligation, splint ligation of an adaptor molecule having a 5′ ssDNA overhang, and split ligation of an adaptor molecule having a 3′ ssDNA overhang. In one embodiment, the adaptor molecule comprises a 5′ ssDNA overhang comprising at least 2 random nucleotides at the 5′ end of the 5′ overhang. In one embodiment, the adaptor molecule comprises a 3′ ssDNA overhang comprising at least 2 random nucleotides at the 3′ end of the 3′ overhang.

In one embodiment, the nucleic acid molecule is crosslinked to the protein of interest, and the method further comprises a step of reversing the crosslinks after ligation of a first adaptor molecule.

In one embodiment, the invention relates to a method for identifying a binding site of a protein of interest, the method comprising the steps of: a) immunoprecipitating the protein of interest bound to a nucleic acid molecule, b) contacting the immunoprecipitated nucleic acid molecule with at least one transposase bound to an adaptor molecule, c) washing the immunoprecipitated nucleic acid molecule at least once with a chaotrophic wash buffer, d) contacting the immunoprecipitated nucleic acid molecule with least one 5′→3′ exonuclease to generate a single-stranded nucleic acid region on the nucleic acid molecule, e) eluting the nucleic acid molecule, f) contacting the eluted nucleic acid molecule with a non-specific primer and a polymerase for primer extension to generate a dsDNA molecule, g) performing A-tailing on the eluted nucleic acid molecule, h) ligating a second adaptor molecule to the eluted nucleic acid molecule, i) amplifying the eluted nucleic acid molecule, and j) sequencing the amplified products.

In one embodiment, step h) comprises ligating the second adaptor molecule by a method selected from the group consisting of tagmentation, 5′ ssDNA ligation, 3′ ssDNA ligation, splint ligation of an adaptor molecule having a 5′ ssDNA overhang, and split ligation of an adaptor molecule having a 3′ ssDNA overhang. In one embodiment, the adaptor molecule comprises a 5′ ssDNA overhang comprising at least 2 random nucleotides at the 5′ end of the 5′ overhang. In one embodiment, the adaptor molecule comprises a 3′ ssDNA overhang comprising at least 2 random nucleotides at the 3′ end of the 3′ overhang.

In one embodiment, the transposase is a hyperactive Tn5 with reduced target sequence specificity.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1C, depicts exemplary experimental results demonstrating an evaluation of ChIP-Nexus data. FIG. 1A depicts a schematic of a completed ChIP-Nexus DNA library. FIG. 1B depicts exemplary experimental results demonstrating the nucleotide frequency at the 5′ end of the sequencing tags among tags that pass (left) or fail (right) the computational filter as defined previously (He et al., (2015) Nat Biotechnol, 33:395-401). Without being bound by theory, FIG. 1C depicts a proposed explanation for the pattern of nucleotide frequency observed in tags that fail to pass filter. Desired end-trimming produces blunt-end DNA as shown in steps 3 and 4a. Excessive trimming will produce a 5′ overhang in step 4b that would result in the pattern at the sequenced tag seen in FIG. 1B.

FIG. 2, comprising FIG. 2A through FIG. 2B, depicts exemplary experimental results demonstrating purification of hyperactive Tn5. FIG. 2A depicts an exemplary SDS-PAGE gel of fractions collected during Tn5 purification. Heparin fractions #12 to #14 were combined and dialyzed for the final prep. The expected size of His6-tagged Tn5 is 54 kilodaltons. Molecular weight markers are shown in lane 1. FIG. 2B depicts a schematic comparing the first steps of ChIP-exo 1.0/1.1 to ChIP-exo 3.0.

FIG. 3, comprising FIG. 3A through FIG. 3C, depicts exemplary experimental results demonstrating that ChIP-exo 3.0 library formation requires a high-stringency wash to remove spent Tn5. FIG. 3A depicts an exemplary 2% agarose gel of the library prep following 18 cycles of PCR for Reb1-TAP samples testing multiple versions of tagmentation-based assays. FIG. 3B depicts an exemplary gel of Abf1-TAP ChIP-exo 3.0 libraries that included a guanidine wash buffer following the tagmentation reaction. FIG. 3C depicts an exemplary gel of Reb1-TAP ChIP-exo 3.0 libraries that included various wash buffers following the tagmentation reaction.

FIG. 4, comprising FIG. 4A through FIG. 4C, depicts exemplary experimental results demonstrating a comparison of yeast transcription factors across ChIP-exo assay versions. FIG. 4A depicts exemplary heatmaps of the top 200 Abf1 motifs for two ChIP-seq and five ChIP-exo versions. FIG. 4B depicts exemplary heatmaps of the top 975 Reb1 primary motifs for two ChIP-seq and five ChIP-exo versions. FIG. 4C depicts exemplary heatmaps of the top 200 Ume6 motifs in 200 bp windows for two ChIP-seq and five ChIP-exo versions. Rows are linked between factors. Each are sorted by the ChIP-exo 5.0 dataset.

FIG. 5, comprising FIG. 5A through FIG. 5E, depicts exemplary experimental results demonstrating that Tn5-based ChIP assays produce a high degree of sequence bias in reads. FIG. 5A depicts exemplary heatmaps comparing assay variants at the top 200 S. cerevisiae Ume6 motifs in a 200 bp (top) or 2 kb (bottom) window. Rows are linked and sorted (in all figures) based on motif-associated tag intensity derived from ChIP-exo 5.0. The data in FIG. 5A contains a subset of the data presented in FIG. 4C. FIG. 5B depicts an exemplary frequency distribution plot of library insert sizes determined by paired-end sequencing for assay version shown in FIG. 5A. Dotted lines indicates the modal insert size within each dataset. The numbers in parentheses represent the insert size mode plus/minus one standard deviation. Without being bound by theory, FIG. 5C depicts a proposed model of multi-tagmented DNA in ChIPmentation. Following tagmentation, Tn5 (spheres) do not dissociate, allowing the excess cut DNA (upper lines) to remain noncovalently bound to the ChIPped DNA (lower lines with sphere attached to the “Y” representing an antibody) through the end of library prep. This results in a mixture of forward and reverse strand tags at the original tagmentation sites that appear purple when viewed in a heatmap (purple asterisks). FIG. 5D depicts an exemplary plot of nucleotide frequency at the 5′ end of Read_2 sequencing tags (and thus not exonuclease digested) generated through tagmentation. Dotted lines indicate the background nucleotide frequency of A/T (31% each) and G/C (19% each) content in S. cerevisiae. The observed sequence bias is displayed above the graph (IUPAC nomenclature). A sequence was deemed biased if the nucleotide frequency was more than 10% above background (A/T>34% or G/C>21%). The Tn5 insertional target recognition sequence (SEQ ID NO:1) (Goryshin et al., (1998) Proc Natl Acad Sci USA, 95:10716-10721) is displayed above the observed sequence bias (SEQ ID NO:2). Asterisks indicate positions that are consistent between the two sequences. FIG. 5E depicts an exemplary plot of nucleotide frequency at the 5′ end of Read_1 sequencing tags generated through exonuclease digestion. Exonuclease treatment masks the bias seen in FIG. 5D, which is still present when considering tag yield (occupancy).

FIG. 6, comprising FIG. 6A through FIG. 6C, depicts exemplary experimental results demonstrating that a comparison to ChIP-exo 1.1 reveals shouldering observed in Tn5-based ChIP-assays (ChIP-exo 3.0 and ChIPmentation). FIG. 6A depicts an exemplary composite plot comparing ChIP-exo 1.1 and 3.0 in a 1 kb window (left) or zoomed in to 200 bp at the top 200 Ume6 motifs. ChIP-exo 3.0 contains more tags that map hundreds of bp away from the binding site than ChIP-exo 1.1. The same high-resolution peaks are captured by both assays. FIG. 6B depicts an exemplary composite plot comparing ChIPmentation and ChIP-exo 3.0 in a 1 kb window (left) or zoomed in to 200 bp at the top 200 Ume6 motifs. The shouldering seen in ChIPmentation and ChIP-exo 3.0 are very similar, but ChIPmentation lacks the high-resolution peaks seen at the binding site. FIG. 6C depicts an exemplary composite plot comparing the pattern generated by the Nextera Tn5 to that of Tn5 prepared in-house as described in the Methods. The top 200 Abf1 sites are shown. Both Tn5 sources produced equivalent shouldering.

FIG. 7, comprising FIG. 7A through FIG. 7B, depicts exemplary experimental results demonstrating that ChIP-exo 4.0 and 4.1 rely on different single-stranded DNA (ssDNA) ligation strategies of adapters having embedded random nucleotide pentamers. FIG. 7A depicts a scheme for ChIP-exo 4.0. FIG. 7B depicts a scheme for ChIP-exo 4.1. These versions of ChIP-exo swap the order in which Read_1 and Read_2 adapters are ligated to the ChIP DNA, and thus involve distinct genomic substrates. In ChIP-exo 4.0, the random pentamer (as one exemplary embodiment) is incorporated immediately 5′ to the exonuclease stop site, thereby shifting the peak of exonuclease stop sites by five bp when using the standard Illumina Read_1 primer. In ChIP-exo 4.1, the random pentamers anneal to the opposite strand, and thus are not incorporated into Read_1 (although are incorporated into Read_2 when conducting paired-end sequencing). Both ChIP-exo 4.0 and ChIP-exo 4.1 involve a second ligation using the same mechanism described for the first ligation of ChIP-exo 4.1, including use of a random pentamer (designated as “NNN” in the schematic).

FIG. 8, comprising FIG. 8A through FIG. 8B, depicts exemplary experimental results demonstrating ChIP-exo optimization. FIG. 8A depicts an exemplary 2% agarose gel of the library preparation following 18 cycles of PCR for Reb1 and Ume6-TAP samples of ChIP-exo 4.0 testing the effect of performing the second adapter ligation on or off resin. FIG. 8B depicts an exemplary gel of ChIP-exo 4.0 libraries testing the effect of T4 DNA polymerase I on DNA polishing. The Abf1 and Ume6-TAP libraries that excluded T4 DNA polymerase I had 2.1 and 2.8-fold higher yield than those with polymerase, respectively.

FIG. 9, comprising FIG. 9A through FIG. 9B, depicts exemplary experimental results demonstrating that ChIP-exo 4.0/4.1 display increased shouldering at the binding site. FIG. 9A depicts exemplary heatmaps comparing assay versions at the top 200 Ume6 motifs in a 200 bp (top) or 2 kb (bottom) window. The data in FIG. 9A contains a subset of the data presented in FIG. 4C. FIG. 9B depicts exemplary composite plots of assay versions in a 1 kb window (left) and zoomed to 200 bp (right). The 1 kb window highlights the increased shouldering observed in ChIP-exo 4.0/4.1. The zoomed view highlights that peaks in ChIP-exo 4.0 are shifted 5 bp away from the motif center due to incorporation of the random pentamer; and the peak observed in ChIP-exo 1.1 at the motif midpoint was absent from the ChIP-exo 4.0/4.1 pattern.

FIG. 10, comprising FIG. 10A through FIG. 10D, depicts exemplary experimental results demonstrating that ChIP-exo 5.0 increases library yield. FIG. 10A depicts a schematic of ChIP-exo 5.0. The purple triangle indicates the location of the Read_1 start site, which is also the λ exonuclease stop site. FIG. 10B depicts exemplary heatmaps comparing ChIP-exo 1.1 and 5.0 at the 975 Reb1 primary motifs in a 200 bp window. Following ChIP, the sample was split and libraries prepared using the indicated protocols. After splitting the sample, each reaction contained a 50 ml cell equivalent of yeast chromatin, which is five-fold less than the amount optimized for ChIP-exo 1.1. FIG. 10C depicts an exemplary composite plot of data from FIG. 10B. FIG. 10D depicts an exemplary 2% agarose gel of the library prep following 18 cycles of PCR for various S. cerevisiae transcription factors using ChIP-exo 1.1 or 5.0. As in FIG. 10B, the samples were split after ChIP. ChIP-exo 5.0 produced greater library yield for all samples.

FIG. 11, comprising FIG. 11A through FIG. 11C, depicts exemplary experimental results demonstrating that ChIP-exo 5.0 produces the same quality data as ChIP-exo 1.1. FIG. 11A depicts exemplary heatmaps comparing ChIP-exo 1.1 and ChIP-exo 5.0 at the top 10,000 H. sapiens CCCTC-binding factor (CTCF) motifs in a 200 bp (top) or 2 kb (bottom) window. FIG. 11B depicts an exemplary comparison of the nucleotide frequency at the 5′ end of the sequencing tags for ChIP-exo 1.1 and ChIP-exo 5.0 in CTCF datasets. Read_1 is the product of exonuclease digestion. Read_2 is the produce of ligation following A-tailing. FIG. 11C depicts exemplary composite plots of data in FIG. 11A in a 1 kb window (left) and zoomed to 200 bp (right).

FIG. 12, comprising FIG. 12A through FIG. 12D, depicts exemplary experimental results demonstrating ChIP-seq 1-step as a simplified version of traditional ChIP-seq. FIG. 12A depicts a schematic of ChIP-seq 1-step, which involves a single enzymatic step in library construction. Although additional adapter sequences are added during PCR, the entire adapter sequence can in principle be included in the ligation step. The possibility of not capturing all possible combinations of frayed ends, which might reduce yield, may be compensated by the overall efficiency of this 1-step library construction. FIG. 12B depicts exemplary heatmaps comparing standard ChIP-seq and ChIP-seq 1-step at the top 10,000 H. sapiens CTCF motifs in a 2 kb window. FIG. 12C depicts exemplary heatmaps comparing ChIP-seq and ChIP-seq 1-step at the 975 S. cerevisiae Reb1 primary motifs (Rhee and Pugh, (2011) Cell, 147:1408-1419) in a 2 kb window. FIG. 12D depicts an exemplary comparison of nucleotide frequency at the 5′ end of the sequencing tags for ChIP-seq and ChIP-seq 1-step in CTCF datasets. In standard ChIP-seq, both ends are first polished, then A-tailed, and then ligated to adapters. Consequently, the sequence bias at both ends is indistinguishable. In ChIP-seq 1-step, the observed bias is dependent on the polarity of the splint ligation.

FIG. 13 depicts a schematic diagram of the nucleic acid molecules used in the various methods of the invention. Each DNA strand and each DNA end is numbered. “X” denotes a blocked 5′ or blocked 3′ end. “p” denotes a 5′ phosphate.

### DETAILED DESCRIPTION

The present invention relates to methods and compositions for detecting the sequence of a nucleic acid binding site of a protein of interest. The methods of the invention have been developed to reduce one or more of the time and reagents of chromatin immunoprecipitation followed by deep sequencing (ChIP-seq) and ChIP-exo. The invention provides multiple improved ChIP-seq and ChIP-exo protocols, each with use-specific advantages. The new versions are greatly simplified through removal of multiple enzymatic steps. This is achieved in part through the use of Tn5 tagmentation and/or single-stranded DNA ligation. The result is greater library yields, lower processing time, and lower cost.

In one embodiment, a modified ChIP-exo method of the invention comprises a step of ligating at least one adaptor molecule to an immunoprecipitated nucleic acid molecule while the nucleic acid molecule remains immobilized. In one embodiment, the method of ligating the at least one adaptor molecule is through tagmentation of the immobilized DNA molecule using a hyperactive transposase with reduced recognition site specificity. In one embodiment, the method of ligating the at least one adaptor molecule is through single stranded DNA (ssDNA) ligation following exonuclease digestion of one strand of an immobilized duplex DNA molecule. In one embodiment, the method of ligating the at least one adaptor molecule is through splint ligation of an adaptor molecule having a random sequence in a ssDNA overhang.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. Microarrays can be prepared and used by a number of methods, including those described in U.S. Pat. No. 5,837,832 (Chee et al.), PCT application WO95/11995 (Chee et al.), Lockhart, D. J. et al. (Nat. Biotech. 14:1675-1680, 1996) and Schena, M. et al. (Proc. Natl. Acad. Sci. 93:10614-10619, 1996), all of which are incorporated herein in their entirety by reference. In other embodiments, such arrays can be produced by the methods described by Brown et al., U.S. Pat. No. 5,807,522.

The terms “comprise(s),” “include(s),” “having,” “has,” “can,” “contain(s),” and variants thereof, as used herein, are intended to be open-ended transitional phrases, terms, or words that do not preclude the possibility of additional acts or structures.

The singular forms “a,” “and” and “the” include plural references unless the context clearly dictates otherwise. The present disclosure also contemplates other embodiments “comprising,” “consisting of” and “consisting essentially of” the embodiments or elements presented herein, whether explicitly set forth or not.

As used herein, an “adaptor” of the present invention means a piece of nucleic acid that is added to a nucleic acid of interest, e.g., the polynucleotide. Two adaptors of the present invention are preferably ligated to the ends of a DNA fragment cross-linked to a polypeptide of interest, with one adaptor on each end of the fragment. Adaptors of the present invention can comprise a primer binding sequence, a random nucleotide sequence, a barcode, or any combination thereof.

“Amplification,” as used herein, refers to any in vitro process for increasing the number of copies of a nucleotide sequence or sequences, i.e., creating an amplification product which may include, by way of example additional target molecules, or target-like molecules or molecules complementary to the target molecule, which molecules are created by virtue of the presence of the target molecule in the sample. These amplification processes include but are not limited to polymerase chain reaction (PCR), multiplex PCR, Rolling Circle PCR, ligase chain reaction (LCR) and the like, in a situation where the target is a nucleic acid, an amplification product can be made enzymatically with DNA or RNA polymerases or transcriptases. Nucleic acid amplification results in the incorporation of nucleotides into DNA or RNA. As used herein, one amplification reaction may consist of many rounds of DNA replication. PCR is an example of a suitable method for DNA amplification. For example, one PCR reaction may consist of 2-40 “cycles” of denaturation and replication.

“Amplification products,” “amplified products” “PCR products” or “amplicons” comprise copies of the target sequence and are generated by hybridization and extension of an amplification primer. This term refers to both single stranded and double stranded amplification primer extension products which contain a copy of the original target sequence, including intermediates of the amplification reaction.

As used herein, an “antibody” encompasses naturally occurring immunoglobulins, fragments thereof, as well as non-naturally occurring immunoglobulins, including, for example, single chain antibodies, chimeric antibodies (e.g., humanized murine antibodies), heteroconjugate antibodies (e.g., bispecific antibodies). Fragments of antibodies include those that bind antigen, (e.g., Fab′, F(ab′)2, Fab, Fv, and rlgG). See, e.g., Pierce Catalog and Handbook, 1994-1995 (Pierce Chemical Co., Rockford, III.); Kuby, J., Immunology, 3rd Ed., W.H. Freeman & Co., New York (1998). The term “antibody” further includes both polyclonal and monoclonal antibodies.

“Appropriate hybridization conditions” as used herein may mean conditions under which a first nucleic acid sequence (e.g., primer, etc.) will hybridize to a second nucleic acid sequence (e.g., target, etc.), such as, for example, in a complex mixture of nucleic acids. Appropriate hybridization conditions are sequence-dependent and will be different in different circumstances. In one embodiment, an appropriate hybridization conditions may be selective or specific wherein a condition is selected to be about 5-10° C. lower than the thermal melting point (Tm) for the specific sequence at a defined ionic strength and pH. In one embodiment, an appropriate hybridization condition encompasses hybridization that occurs over a range of temperatures from more to less stringent. In one embodiment, a hybridization range may encompass hybridization that occurs from 98° C. to 50° C. According to the invention, such a hybridization range may be used to allow hybridization of the primers of the invention to target sequences with reduced specificity, for the purposes of amplifying a broad range of nucleic acid molecules with a single set of primers.

A “barcode”, as used herein, refers to a nucleotide sequence that serves as a means of identification for sequenced polynucleotides of the present invention. Barcodes of the present invention may comprise at least 4 random bases, such as 5, 6, 7, 8, 9, 10, 11, 12, 13, 14, 15, 16, 17, 18, 19, 20 or more bases in length. Alternatively, or in addition to the random nucleotides, the barcode may have three or more fixed bases, 4, 5, 6, 7, 8, 9, 10, 11, 12, 13, 14, 15, 16, 17, 18, 19, 20 or more bases in length. In some embodiments, both random and fixed bases are used as barcodes. For example, a barcode can be composed of 5 random bases and 4 fixed bases. Methods for designing barcodes are known in the art. See, e.g., Bystrykh (2012) PLoS ONE, 7(5): e36852; Mir et al., (2013) PLoS ONE, 8(12): e82933.

As used herein, “binding” means an association interaction between two molecules, via covalent or non-covalent interactions including, but not limited to, hydrogen bonding, hydrophobic interactions, van der Waals interactions, and electrostatic interactions. Binding may be sequence specific or non-sequence specific. Non-sequence specific binding may occur when, for example, a polypeptide of interest (i.e. a histone) binds to a polynucleotide of any sequence. Specific binding may occur when, for example, a polypeptide of interest (i.e. a transcription factor) binds oredominantly to a highly restricted sequence of nucleotides.

As used herein, a “chromatin immunoprecipitation-exonuclease (ChlP-exo) process” means a protocol wherein an antibody to the protein of interest is used to isolate a plurality of polypeptide of interest-polynucleotide complexes following which the complexes are exposed to exonuclease digestion, resulting in digestion of the bound polynucleotide up to the site of protection by the polypeptide of interest, such that the polynucleotide that represent at least one location in the polynucleotide at which the polypeptide of interest binds.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

As used herein, “dA tailing” the polynucleotide fragment means a protocol in which 3′ deoxyadenine (dA) tails are added to a polynucleotide.

As used herein, “digesting” refers to the enzymatic removal of nucleotides from a polynucleotide.

As used herein, “dNTPs” refers to a mixture of different deoxyribonucleotide triphosphates: deoxyadenosine triphosphate (dATP), deoxycytidine triphosphate (dCTP), deoxyguanosine triphosphate (dGTP) and deoxythymidine triphosphate (dTTP).

As used herein, “eluting” the polynucleotide fragment-polypeptide of interest complexes from the substrate refers to a protocol in which an elution buffer is incubated with substrate-linked polynucleotide fragment-polypeptide of interest complexes to separate the complexes from the substrate.

As used herein, “end repairing” the polynucleotide fragment means a protocol in which fragmented DNA, for example, produced by shearing, nuclease treatment or ligation, is processed to generate blunt-ended dsDNA fragments with 5′ phosphorylated ends on both of the strands. Details for end repair reactions are well known and are disclosed herein or may be found in e.g., Evans et al., 2008.

“Fragment” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. When comparing DNA and RNA, thymine (T) and uracil (U) may be considered equivalent. Identity may be performed manually or by using a computer sequence algorithm such as BLAST or BLAST 2.0.

As used herein, “immunoprecipitating”, and grammatical variations thereof, refers to a protocol in which polypeptides, such as antibodies, that specifically bind target polypeptides, are utilized to separate the target polypeptides and the substances that are physically linked to such polypeptides (such as a polynucleotide) from a plurality of other cellular materials. For example, cross-linked polypeptide-polynucleotide complexes of the present invention may be separated from other cellular materials by applying a cell extract to an affinity purification matrix, wherein the affinity purification matrix comprises an antibody specific for the target polypeptide linked to a substrate. The target polypeptide-polynucleotide complexes will bind to the antibody and may later be eluted, thereby separating the target polypeptide-polynucleotide complexes from other cellular materials. Detailed conditions for immunoprecipitation are disclosed herein and are also known in the art and may be found in e.g., Bonifacino et al., (2016) Curr Protoc Cell Biol, 71:7.2.1-7.2.24.

A “Klenow fragment” of the present invention refers to a fragment of E. coli DNA polymerase I that has been enzymatically processed to be capable of 5′-3′ polymerase activity and 3′-5′ exonuclease activity. Preferably, a Klenow fragment of the present invention is not capable of 3′-5′ exonuclease activity (3′-5′ exo”).

As used herein, “ligating” means the joining of the 5′ and the 3′ end of the same DNA molecule or two different DNA molecules. The former reaction results in a circular DNA molecule whereas the latter produces a linear DNA molecule. Ligases of the present invention may include T4 DNA Ligase, T7 DNA Ligase, CircLigase, transposases and others known to those of skill in the art. Ligation reactions include, but are not limited to, sticky end ligations, transposase-mediated ligations and blunt end ligations. Sticky end ligations involve complementary “overhangs” wherein one DNA strand of a mostly dsDNA molecule comprises non-base paired nucleotides at the end of the molecule. Such non-base paired nucleotides may base pair with complementary non-base paired nucleotides on the same or a different DNA molecule, enabling a ligase to catalyze the covalent linkage of the ends of the DNA molecule(s). Blunt end ligations are non-specific ligations that do not involve complementary base pairing. Transposase mediated ligation methods involve a “cut and paste” reaction in which a transposon cleaves a dsDNA molecule and then ligates a nucleic acid sequence onto the cleaved dsDNA ends. Ligation may also be performed on either single stranded or double stranded DNA.

As used herein, a “nuclease” is an enzyme that catalyzes the breakage of phosphodiester bonds connecting the nucleic acid subunits of a polynucleotide. A nuclease of the present invention may be an exonuclease or an endonuclease. Depending on the enzyme, an exonuclease catalyzes breakage of phosphodiester bonds either at the 5′ or at the 3′ end of a polynucleotide, thereby releasing the nucleic acids at the end of the polynucleotide. An endonuclease catalyzes breakage of phosphodiester bonds connecting nucleic acid subunits not found at the ends of a polynucleotide. Nucleases of the present invention, when acting on dsDNA, preferably catalyze breakage of phosphodiester bonds on both strands of the dsDNA. Nucleases may cleave equivalent phosphodiester bonds of complementary base pairs on each strand of a dsDNA molecule, thereby creating, from one dsDNA molecule, two dsDNA fragments with “blunt ends”. Alternatively, nucleases may catalyze cleavage of phosphodiester bonds of non-complementary base pairs, thereby creating, from one dsDNA molecule, two dsDNA fragments with “overhangs” or “sticky ends”.

“Nucleic acid” or “oligonucleotide” or “polynucleotide” or “nucleic acid fragment” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence. Thus, a nucleic acid also encompasses a probe that hybridizes under appropriate hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

As used herein, a “polymerase” means an enzyme that generates polymers of nucleic acids. Preferably, the polymerase is an RNA polymerase or DNA polymerase. A polymerase of the present invention may interact with a genome at any position in the genome. In the case of RNA polymerase, the polymerase interacts with regions of the genome that code for functional products, i.e. genes. Transcription of a given gene in eukaryotes typically does not occur constitutively, but instead requires interaction of a transcription initiation complex, comprising, for example, transcription factors, with enhancer elements, promoter elements, and combinations thereof, in order to recruit a polymerase to a transcription start site.

As used herein, a “polypeptide of interest” may be any polypeptide for which said polypeptide's genomic binding regions are sought. It is envisioned that a polypeptide of the present invention may include full length proteins and protein fragments. While the methods of the present invention may be utilized not only to determine at least one region of a genome at which a polypeptide of interest binds, they may also be utilized to determine if a polypeptide binds to a genome at all. The polypeptide of interest may selected from the group consisting of a transcription factor, a polymerase, a nuclease, and a histone.

As used herein, “precipitating” the polynucleotides of the present invention refers to a process well known to those of skill in the art in which substantially pure polynucleotides in solution are mixed with ethanol to draw the polynucleotides out of solution and into a solid precipitate.

“Primer” as used herein refers to a single-stranded oligonucleotide or a single-stranded polynucleotide that is extended on its 3′ end by covalent addition of nucleotide monomers during amplification. Nucleic acid amplification often is based on nucleic acid synthesis by a nucleic acid polymerase. Many such polymerases require the presence of a primer that can be extended to initiate such nucleic acid synthesis.

As used herein, “purifying” the polynucleotides of the present invention refers to a process well known to those of skill in the art in which polynucleotides are substantially separated from other components in a sample, including, but not limited to, polypeptides of interest.

As used herein, “sample” or “test sample,” may refer to any source used to obtain nucleic acids for examination using the compositions and methods of the invention. A test sample is typically anything suspected of containing a target sequence. Test samples can be prepared using methodologies well known in the art such as by obtaining a specimen from an individual and, if necessary, disrupting any cells contained thereby to release genomic nucleic acids. These test samples include biological samples which can be tested by the methods of the present invention described herein and include human and animal cells, tissues and body fluids such as whole blood, serum, plasma, cerebrospinal fluid, sputum, bronchial washing, bronchial aspirates, urine, lymph fluids and various external secretions of the respiratory, intestinal and genitourinary tracts, tears, saliva, milk, white blood cells, myelomas, buccal cells, cervicovaginal cells, epithelial cells from urine, fetal cells, or any cells present in tissue obtained by biopsy and the like; biological fluids such as cell culture supernatants; tissue specimens which may be fixed; and cell specimens which may be fixed.

Any DNA sample may be used in practicing the present invention, including without limitation eukaryotic, prokaryotic and viral DNA. In one embodiment, the target DNA represents a sample of genomic DNA isolated from a patient. This DNA may be obtained from any cell source, tissue source, or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen and tissue exudates at the site of infection or inflammation. DNA is extracted from the cell source, tissue source, or body fluid using any of the numerous methods that are standard in the art. It will be understood that the particular method used to extract DNA will depend on the nature of the source.

As used herein, “reverse cross-linking” the polypeptide-polynucleotide complex refers to a protocol well known to those of skill in the art in which a protease (i.e., Protease K), heat, or both are utilized to break the covalent linkages between the polypeptides of interest and the polynucleotide fragments.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of about 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under appropriate hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%,or 99% over a region of about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

As used herein, a “substrate” is a solid platform on which antibodies used in immunoprecipitation are bound.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Described herein are methods, systems and kits for identifying the location at which a protein binds a nucleic acid molecule. The methods, systems and kits provide for determination of a binding location to near base-pair resolution (the median resolution is less than 5 by (e.g., 1, 2, 3, 4, 5 bp) for tested sequence-specific DNA binding proteins that occupy their cognate sites at least 5% of the time). A typical method for identifying the location at which a protein binds in a genome includes several steps that are performed in a conventional ChIP-exo assay, but further includes modifications of the ChIP-exo assay that reduce one or more of the time and reagents required for the assay. The conventional ChIP-exo assay is described in U.S. Pat. No. 8,367,334 which is incorporated herein in its entirety.

Although the invention is described in terms of a modified ChIP-seq or modified ChIP-exo method, it should be understood that the methods of the invention can be applied to other immunoprecipitation-based next-Gen sequencing assays including, but not limited to, permanganate/piperidine (PIP-seq), WhIP-exo, PB-exo, MNase ChIP-seq and PB-seq. In addition, it should be understood that the term immunoprecipitation is used to include other forms of affinity purification and therefore the methods of the invention can be applied to methods in which proteins of interest are precipitated using affinity purification methods, including, but not limited to, precipitation of a protein of interest using a purification tag, or through enzymatic modification (e.g., biotinylation). Exemplary purification tags include, but are not limited to, chitin binding protein (CBP), maltose binding protein (MBP), Strep-tag, glutathione-S-transferase (GST), poly(His) tag, FLAG-tag and epitope tags which include, but are not limited to V5-tag, Myc-tag, HA-tag and NE-tag.

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). ChIP methods are known in the art and are described in Pugh and Gilmour, Genome Biology vol. 2(4):reviews 1013.1-1013.3, 2001; Lee et al., Nature Protocols vol. 1(2):729-748, 2006; and Collas and Dahl, Front Biosci. Vol. 13:929-943, 2008, as well as in methodology treatises such as Chromatin Immunoprecipitation Assays: Methods and Protocols (Methods in Molecular Biology) by Philippe Collas, 1st edition, 2009, Humana Press, Totowa, N.J.; and DNA-Protein Interactions (Methods in Molecular Biology) by Tom Moss (ed.) and Benoit Leblanc (ed.), 3rd edition, 2009, Humana Press, Totowa, N.J. Ligation-mediated polymerase chain reaction (LM-PCR) methods are also known in the art and are described, for example, in Ngoc et al., FEMS Microbiol. Lett. Vol. 288:33-39, 2008; and Tagoh et al., Methods Mol. Biol. Vol. 325:285-314, 2006.

Any type of cell or reconstituted protein-nucleic acid complex can be used in the modified ChIP-seq or modified ChIP-exo assays of the invention. Any sample from which nucleic acid molecules can be isolated can be used in the assay system. Indeed, in certain instances it may be advantageous to use different sample types, e.g., blood, cancer cells, saliva, and formalin-fixed paraffin embedded (FFPE) samples.

The assays are also applicable in the absence of crosslinking, as long as the protein remains bound to the nucleic acid. A population of cells (or in vitro assembled complexes) is incubated with a chemical crosslinking reagent such as formaldehyde, which crosslinks proteins to each other and to nucleic acids such as DNA and RNA. Any suitable crosslinking reagent can be used. In one embodiment, the crosslinker is used to preserve in vivo protein-nucleic acid interactions during the stringent work-up conditions that are meant to diminish nonspecific contamination. The crosslinking reaction is almost instantaneous, and provides a snapshot of the protein-nucleic acid interactions taking place in the cell. The next step of the assay requires cell disruption and washing of the insoluble chromatin to remove non-chromatin soluble proteins. In one embodiment, the chromatin is then fragmented and solubilized using sonication. Sonication randomly shears DNA to a size range of about 300 by in yeast and 0.5-1 kb in vertebrates, although more intense sonication can create smaller fragment sizes.

In one embodiment the modified ChIP-seq or modified ChIP-exo assays of the invention include purification a chromatin/nucleic acid binding protein, typically in the form of immunoprecipitation where an immobilized antibody against the protein is used to selectively pull out of solution the target protein. Along with the immunopurified protein comes any nucleic acid to which it is crosslinked. Buffer and wash conditions are of sufficient stringency (usually with low levels of the detergent SDS) that retention of nucleic acid contaminants that have not been directly or indirectly crosslinked to the target protein are diminished but not eliminated.

In various embodiments of the modified ChIP-seq and ChIP-exo assays of the invention, the ends of the fragmented complexed are ligated or annealed to a known DNA sequence such as a DNA adaptor prior to crosslink reversal, then later sequencing of the DNA fragment will allow the end at the crosslinked barrier to be distinguished from the other end generated during fragmentation by sonication.

In various embodiments, the adapters that are added to the 5′ and/or 3′ end of a nucleic acid can comprise a universal sequence. A universal sequence is a region of nucleotide sequence that is common to, i.e., shared by, two or more nucleic acid molecules.

Optionally, the two or more nucleic acid molecules also have regions of sequence differences. Thus, for example, the 5′ adapters can comprise identical or universal nucleic acid sequences and the 3′ adapters can comprise identical or universal sequences. A universal sequence that may be present in different members of a plurality of nucleic acid molecules can allow the replication or amplification of multiple different sequences using a single universal primer that is complementary to the universal sequence.

In one embodiment, the adaptor molecule comprises a sequence containing a plurality of random nucleotides at the 5′-terminus or 3′-terminus. In various embodiments, the plurality of random nucleotides are present in a single-stranded region of the adaptor molecule. In one embodiment, the adaptor molecule comprises at least 2, at least 3, at least 4, at least 5, at least 6, at least 7, at least 8, at least 9, or at least 10 random nucleotides.

Exonucleases having 5′-3′ single-stranded- or double-stranded-specific exonuclease activity that can be used in any of the methods of the invention include, but are not limited to, lambda exonuclease, T7 exonuclease, T5 exonuclease, exonuclease II, exonuclease VIII, CCR4-NOT complex, RecJ_{f} exonuclease, exonuclease I, and exonuclease VII. Preferably, the exonuclease having 5′-3′ double-stranded-DNA-specific exonuclease activity is lambda exonuclease, and the exonuclease having 5′-3′ single-stranded-DNA-specific exonuclease activity is RecJ_{f} exonuclease. Lambda exonuclease (as one example of a potential strand-specific exonuclease) catalyzes the 5′-to-3′ removal of 5′ mononucleotides from duplex DNA, leaving the complementary sequence intact.

Any procedures known in the art may be employed for digestion of a single nucleic acid strand of a duplex nucleic acid molecule. In one embodiment, the method for exonuclease digestion includes contacting the immunoprecipitated chromatin fragments with an exonuclease and an appropriate exonuclease buffer for a period of time sufficient for the exonuclease to digest a single nucleic acid strand of a duplex nucleic acid molecule. In one embodiment, the method includes contacting the immunoprecipitated chromatin fragments with λ exonuclease, λ exonuclease reaction buffer, Triton-X 100, and DMSO and incubating the reaction at 37° C. for at least 5 minutes, at least 10 minutes, at least 15 minutes, at least 20 minutes, at least 25 minutes, at least 30 minutes, at least 1 hour, at least 2, hours, at least 3 hours, at least 4 hours, at least 5 hours, at least 6 hours, or for more than 6 hours. In one embodiment, the digestion is followed by one or more washes. In one embodiment, the digestion is followed by a wash with Tris-HCl, pH 8.0 at 4° C.

In various embodiments, one or more polymerases are used in the methods of the invention for steps including, but not limited to, A-tailing of a nucleic acid molecule, end repair of a nucleic acid molecule to generate blund ended dsDNA, primer extension, polymerase chain reaction (PCR), LM-PCR, end trimming, gap filling, end polishing, and polymerase fill-in. DNA polymerases that can be used in the methods of the present invention include, but are not limited to, T4 DNA polymerase, DNA polymerase I, Klenow fragment, phi29 DNA polymerase, Phusion polymerase, and Phusion Hot Start polymerase.

Any procedures known in the art may be employed for A-tailing of a nucleic acid molecule. In one embodiment, the method for A-tailing includes contacting the immunoprecipitated chromatin fragments with a polymerase lacking exonuclease activity, dATP and an appropriate buffer for a period of time sufficient for the polymerase to attach at least one dATP nucleotide onto a 3′ end of a nucleic acid molecule. In one embodiment, the method includes contacting the immunoprecipitated chromatin fragments with Klenow Fragment −exo, NEBuffer 2, and dATP and incubating the reaction at 37° C. for at least 5 minutes, at least 10 minutes, at least 15 minutes, at least 20 minutes, at least 25 minutes, at least 30 minutes, at least 1 hour, at least 2, hours, at least 3 hours, at least 4 hours, at least 5 hours, at least 6 hours, or for more than 6 hours. In one embodiment, the A-tailing is followed by one or more washes. In one embodiment, the A-tailing is followed by a wash with Tris-HCl, pH 8.0 at 4° C.

Any procedures known in the art may be employed for end repair of a nucleic acid molecule. In one embodiment, the method for end repair includes contacting the immunoprecipitated chromatin fragments with a polymerase and an appropriate buffer for a period of time sufficient for the polymerase to attach at least one nucleotide onto a 3′ end of a nucleic acid molecule. In one embodiment, the method includes contacting the immunoprecipitated chromatin fragments with T4 DNA polymerase, DNA Polymerase I, T4 PNK, T4 DNA Ligase Buffer, and dNTPs and incubating the reaction at 12° C. for at least 5 minutes, at least 10 minutes, at least 15 minutes, at least 20 minutes, at least 25 minutes, at least 30 minutes, at least 1 hour, at least 2, hours, at least 3 hours, at least 4 hours, at least 5 hours, at least 6 hours, or for more than 6 hours. In one embodiment, the end repair is followed by one or more washes. In one embodiment, the end repair is followed by a wash with Tris-HCl, pH 8.0 at 4° C.

Any procedures known in the art may be employed for polymerase fill-in of a nucleic acid molecule. In one embodiment, the method for polymerase fill-in includes contacting the immunoprecipitated chromatin fragments with a polymerase and an appropriate buffer for a period of time sufficient for the polymerase to attach at least one nucleotide onto a 3′ end of a nucleic acid molecule. In one embodiment, the method includes contacting the immunoprecipitated chromatin fragments with phi29 polymerase, phi29 reaction buffer, bovine serum albumin (BSA) and dNTPs and incubating the reaction at 30° C. for at least 5 minutes, at least 10 minutes, at least 15 minutes, at least 20 minutes, at least 25 minutes, at least 30 minutes, at least 1 hour, at least 2, hours, at least 3 hours, at least 4 hours, at least 5 hours, at least 6 hours, or for more than 6 hours. In one embodiment, the polymerase fill-in is followed by one or more washes. In one embodiment, the polymerase fill-in is followed by a wash with Tris-HCl, pH 8.0 at 4° C.

In various embodiments, one or more polynucleotide kinases (PNK) are used in the methods of the invention for steps including, but not limited to, phosphorylating a 5′ end of a nucleic acid molecule using a kinase reaction, and end repair. Polynucleotide kinases of the present invention include, but are not limited to, T4 polynucleotide kinase.

Any procedures known in the art may be employed for phosphorylating a 5′ end of a nucleic acid molecule. In one embodiment, the method for phosphorylating a 5′ end of a nucleic acid molecule includes contacting the immunoprecipitated chromatin fragments with a PNK and an appropriate buffer for a period of time sufficient for the PNK to phosphorylate a 5′ end of a nucleic acid molecule. In one embodiment, the method includes contacting the immunoprecipitated chromatin fragments with T4 PNK, T4 DNA Ligase Buffer and BSA and incubating the reaction at 37° C. for at least 5 minutes, at least 10 minutes, at least 15 minutes, at least 20 minutes, at least 25 minutes, at least 30 minutes, at least 1 hour, at least 2, hours, at least 3 hours, at least 4 hours, at least 5 hours, at least 6 hours, or for more than 6 hours. In one embodiment, the polymerase fill-in is followed by one or more washes. In one embodiment, the polymerase fill-in is followed by a wash with Tris-HCl, pH 8.0 at 4° C.

In various embodiments, one or more ligases are used in the methods of the invention for steps including, but not limited to, adaptor ligation, splint ligation, 3′ ssDNA ligation, 5′ ssDNA ligation, and self-circularization of single-stranded (ss) DNA. DNA ligases of the present invention include, but are not limited to, T4 DNA ligase, Quick T4 DNA ligase, and CircLigase.

It is to be understood that exonucleases, DNA polymerases, polynucleotide kinases and DNA ligases are well known to those of skill in the art and that the preceding lists should not be construed as limiting in any way.

In one embodiment, the nucleic acid molecules are bound but not crosslinked to the immunoprecipitated proteins of interest, therefore the modified ChIP-exo and modified ChIP-seq methods of the invention include a step of eluting the bound nucleic acid molecules. Any procedures known in the art that disrupt protein nucleic acid complexes and elute the nucleic acid molecules may be employed.

In one embodiment, the modified ChIP-exo and modified ChIP-seq methods of the invention include a step to reverse the crosslink of a nucleic acid molecule:protein complex, and eluting the nucleic acid molecules. Any procedures known in the art may be employed that reverse the crosslinks and elute the nucleic acid molecules. An exemplary method for reversal of crosslinkes includes incubation of the immunoprecipitated chromatin fragments at a temperature of at least 15° C., at least 20° C., at least 25° C., at least 30° C., at least 35° C., at least 40° C., at least 45° C., at least 50° C., at least 55° C., at least 60° C., or at least 65° C. for at least 30 minutes, at least 1 hour, at least 2, hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24, or for more than 24 hours. An alternative exemplary method for reversal of crosslinkes includes incubation of the immunoprecipitated chromatin fragments at a temperature of at least 80° C., at least 85° C., at least 90° C., or at least 95° C. for at least 10 minutes, at least 15 minutes, at least 20 minutes, at least 25 minutes, at least 30 minutes, or for more than 30 minutes. In one embodiment, the elution and/or crosslink reversal is performed in the presence of one or more of Proteinase K and RNAse. In one embodiment, the nucleic acid molecules are incubated in the presence of one or more of Proteinase K and RNAse prior to or subsequent to elution and/or crosslink reversal.

In one embodiment, the methods of the invention include one or more purification steps. Any procedures known in the art may be employed for purifying a nucleic acid molecule. Methods for purifying a nucleic acid molecule include, but are not limited to, ethanol purification, column-based purification methods, gel-based purification methods, and magnetic bead based purification methods.

In one embodiment, the eluted nucleic acid molecules are amplified prior to sequencing. Any procedures known in the art may be employed that amplify the nucleic acid molecules. An exemplary method for amplification of nucleic acid molecules is using PCR.

In some embodiments, multiple modified ChIP-seq or modified ChIP-exo libraries are sequenced using single-molecule DNA sequencing (either true single molecule or clusters of identical clones) to identify the nucleotide sequences of the individual DNA molecules. In various embodiments, the sequencing can be accommodated by Illumina, Applied Biosystems, Roche, and other deep sequencing technologies. Hybridization-based detection platforms could also be used but provide less resolution.

In some embodiments, multiple modified ChIP-seq or modified ChIP-exo libraries are prepared in parallel and then pooled to generate a high throughput assay. For example, parallel assays may be carried out in a multi-well plate, such as a 96-well plate or a 384 well plate. The number of pooled samples is not necessarily limited as the limiting factors are 1) the number of sequence specific barcodes and 2) the number of sequencing reads desired per sample for a given sequencing platform. Therefore, the method may be extended to include more samples at a cost of reduced sequencing read coverage per sample. Separate sequencing of individual DNA molecules that are truncated at either the right or left border of the protein-DNA crosslink can be used to identify the right and left borders (i.e. left border on “+” vs. right border on “−” strand) of the bound protein. The “footprint” size is determined by the number of base pairs between the left and right borders of the bound protein. In addition, the relative amount of protein binding is determined by the normalized number of sequencing reads clustered under the detected peak. GeneTrack is one means for peak detection and to generate a genome-wide browser of the tag distribution (Albert et al., Bioinformatics, 2008). However, the UCSC browser and any other peak detection method may suffice. GeneTrack software was previously developed for such a purpose, and its use has been reported in several publications (Albert et al., Bioinformatics, 2008; Mavrich et al., Genome Res., 18:1073-1083, 2008; Mavrich et al., Nature, 453:358-362, 2008).

ChIP-Exo 3.0

In one embodiment, the sequencing adaptors are ligated to the target DNA molecule through the process of tagmentation, which is described in detail below. In some embodiments, tagmentation can be used in a ChIP-exo method for generating a library of tagged chromatin fragments for use as next-generation sequencing or amplification templates.

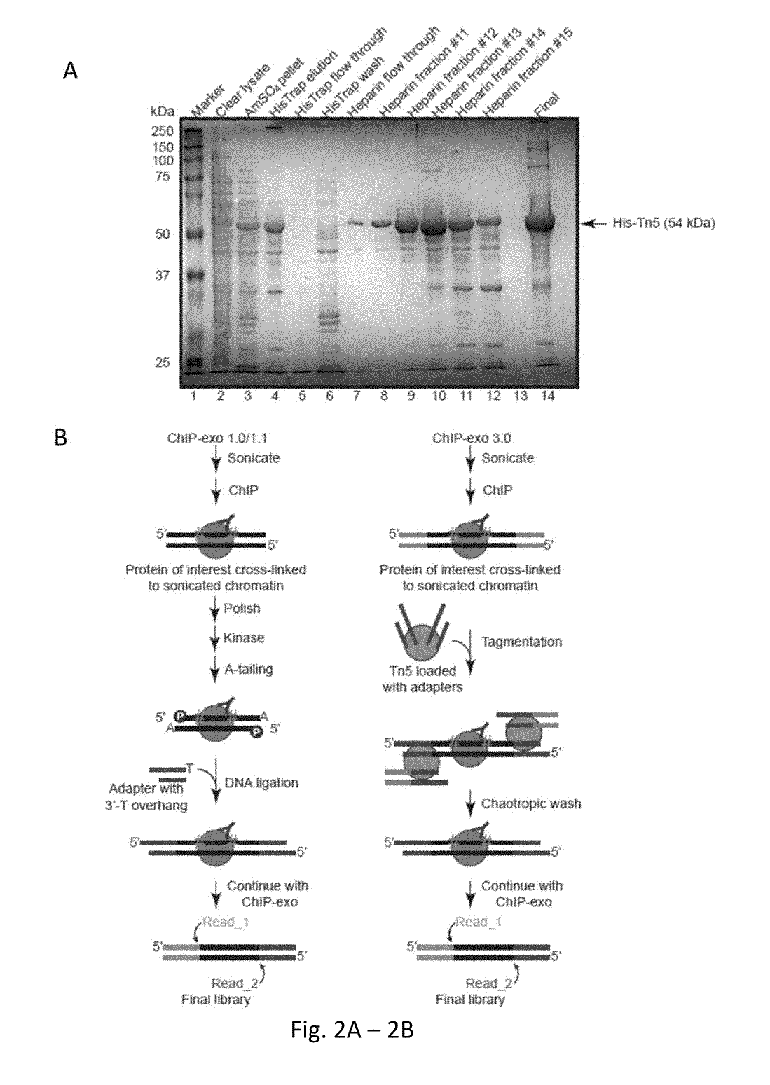

The overall procedure for this method, which is referred to as ChIP-exo 3.0, is depicted in the right hand column of FIG. 2B. In one embodiment, the method comprises the steps of: a) immunoprecipitating the protein of interest bound to a nucleic acid molecule, b) contacting the immunoprecipitated nucleic acid molecule with at least one transposase bound to an adaptor molecule, c) washing the immunoprecipitated nucleic acid molecule at least once with a chaotrophic wash buffer that leaves the crosslinked protein-nucleic acid complex attached to the immobilized antibody, and d) contacting the immunoprecipitated nucleic acid molecule with least one 5′→3′ exonuclease to generate a single-stranded nucleic acid region on the nucleic acid molecule.

In the ChIP-exo 3.0 assay, cells are crosslinked with formaldehyde and lysed. The sample then remains on the resin during the tagmentation and exonuclease digestion steps.

Adaptor molecules are then ligated to the immunoprecipitated chromatin fragment using a tagmentation method. As used herein, the term “tagmentation” refers to the modification of DNA by a transposome complex comprising transposase enzyme complexed with adaptors comprising transposon end sequence. Tagmentation results in the simultaneous fragmentation of the DNA and ligation of the adaptors to the 5′ ends of both strands of duplex fragments. Following a purification step to remove the transposase enzyme, additional sequences can be added to or removed from the ends of the adapted fragments, for example by PCR, ligation, exonuclease digestion or any other suitable methodology known to those of skill in the art.

The method of the invention can use any transposase that can accept a transposase end sequence and cleave a target nucleic acid, attaching a transferred end. A “transposome” is comprised of at least a transposase and a transposase recognition site. In some such systems, the transposase can form a functional complex with a transposon recognition site that is capable of catalyzing a transposition reaction. The transposase or integrase may bind to the transposase recognition site and insert the transposase recognition site into a target nucleic acid in a process sometimes termed “tagmentation”. In some such insertion events, one strand of the transposase recognition site may be transferred into the target nucleic acid.

The number of steps required to transform DNA into adaptor-modified templates in solution ready for cluster formation and sequencing can be minimized by the use of transposase mediated fragmentation and tagging.

In some embodiments, transposon-based technology can be utilized for fragmenting DNA, for example as exemplified in the workflow for Nextera™ DNA sample preparation kits (Illumina, Inc.) wherein genomic DNA can be fragmented by an engineered transposome that simultaneously fragments and tags input DNA (“tagmentation”) thereby creating a population of fragmented nucleic acid molecules which comprise unique adapter sequences at the ends of the fragments.

In one embodiment, the chromatin is first fragmented by sonication, then immunoprecipitated and tagmented while on the resin. In another embodiment, the transposase recognition sequence has been incorporated into the Illumina Nextera sequencing adapters. The transposase inserts one end of each recognition sequence into essentially unfragmented genomic DNA, which fragments the chromatin.

Some embodiments can include the use of a hyperactive Tn5 transposase and a Tn5-type transposase recognition site (Goryshin et al., (1998) J Biol Chem, 273:7367-7374). In one embodiment, the tagmentation method uses a hyperactive Tn5 that binds normally to its 19 bp recognition sequence, but has less sequence specificity for insertional targeting (Reznikoff, (2003) Mol Microbiol, 47:1199-1206). In one embodiment, the tagmentation method may use MuA transposase and a Mu transposase recognition site comprising R1 and R2 end sequences (Mizuuchi, K., Cell, 35: 785, 1983; Savilahti, H, et al, EMBO J., 14: 4893, 1995). More examples of transposition systems that can be used with certain embodiments provided herein include Staphylococcus aureus Tn552 (Colegio et al., J. Bacterid., 183: 2384-8, 2001; Kirby C et al., Mol. Microbiol, 43: 173-86, 2002), Tyl (Devine & Boeke, Nucleic Acids Res., 22: 3765-72, 1994 and International Publication WO 95/23875), Transposon Tn7 (Craig, N L, Science. 271: 1512, 1996; Craig, N L, Review in: Curr Top Microbiol Immunol, 204:27-48, 1996), Tn/O and IS 10 (Kleckner N, et al, Curr Top Microbiol Immunol, 204:49-82, 1996), Mariner transposase (Lampe D J, et al, EMBO J., 15: 5470-9, 1996), Tel (Plasterk R H, Curr. Topics Microbiol. Immunol, 204: 125-43, 1996), P Element (Gloor, G B, Methods Mol. Biol, 260: 97-114, 2004), Tn3 (Ichikawa & Ohtsubo, J Biol. Chem. 265: 18829-32, 1990), bacterial insertion sequences (Ohtsubo & Sekine, Curr. Top. Microbiol. Immunol. 204: 1-26, 1996), retroviruses (Brown, et al, Proc Natl Acad Sci USA, 86:2525-9, 1989), and retrotransposon of yeast (Boeke & Corces, Annu Rev Microbiol. 43:403-34, 1989). More examples include ISS, TnlO, Tn903, IS911, and engineered versions of transposase family enzymes (Zhang et al, (2009) PLoS Genet. 5:e1000689. Epub 2009 Oct. 16; Wilson C. et al (2007) J. Microbiol. Methods 71:332-5).

A “transposition reaction” is a reaction wherein one or more transposons are inserted into target nucleic acids at random sites or almost random sites. Essential components in a transposition reaction are a transposase and DNA oligonucleotides that exhibit the nucleotide sequences of a transposon, including the transferred transposon sequence and its complement (i.e., the non-transferred transposon end sequence) as well as other components needed to form a functional transposition or transposome complex. The DNA oligonucleotides can further comprise additional sequences (e.g., adaptor or primer sequences) as needed or desired.

Following tagmentation, the spent transposase that remained bound to the fragmented chromatin DNA product must be removed, while maintaining the protein-DNA cross-links and protein-antibody interaction. In one embodiment, the spent transposase is removed by washing with a chaotrophic wash buffer. In one embodiment, the chaotrophic wash buffer is a mixed micelle wash buffer, RIPA buffer, FA lysis buffer containing 0.1%, 0.2%, or 0.5% SDS, or a guanidine hydrochloride buffer.

Following removal of the transposase enzyme, the crosslinked nucleic acid molecule is end repaired to generate blunt ends prior to exonuclease digestion. In one embodiment, a double-strand specific 5′→3′ single-stranded exonuclease (e.g. lambda exonuclease) is used to digest one DNA strand up to the bound protein.

In one embodiment, a step of contacting the resin-bound crosslinked molecules with a 5′-to-3′ single-stranded exonuclease (ssExo) is included in the method to digest any contaminating ssDNA. An exemplary 5′-to-3′ ssExo that can be used in the method of the invention includes, but is not limited to, RecJ_{f}. Double stranded DNA is resistant to this exonuclease, and thus this enzyme removes contaminating uncrosslinked single-stranded nucleic acid molecule.

In one embodiment, after exonuclease digestion, the protein-DNA complex is eluted from the resin, by reversing the crosslinks and a second adaptor molecule is ligated to the eluted nucleic acid molecules.

In one embodiment, the second adaptor ligation step includes annealing a splint adaptor to the nucleic acid fragment using a splint adaptor ligation method. Any appropriate method of ligating a splint adaptor may be used in the method of the invention. In one embodiment, the method comprises the use of T4 DNA ligase to anneal a splint adaptor to the 5′ ends of the eluted nucleic acid molecules.

In one embodiment, the splint adaptor comprises one of a pool of splint adaptors having a dsDNA portion and a single-stranded 5′ overhang which each contain at least 2, at least 3, at least 4, at least 5, at least 6, at least 7, at least 8, at least 9, or at least 10 random nucleotides at the 5′ terminus of the single-stranded 5′ overhang. In one embodiment, the splint adaptor comprises one of a pool of duplexes formed from basepairing of nucleic acid molecules having a sequence as set forth in SEQ ID NO:12 with a pool of nucleic acid molecules having a nucleotide sequence as set forth in SEQ ID NO:14, wherein each of the indicated “N” nucleotides of SEQ ID NO:14 represents any nucleotide.

In one embodiment, the second adaptor molecule is ligated by contacting the eluted nucleic acid molecule with a non-specific primer and a polymerase for primer extension to generate a dsDNA molecule, performing A-tailing on the eluted nucleic acid molecule, and ligating a second adaptor molecule to the eluted nucleic acid molecule.

The resulting eluted single-stranded DNA is annealed with a primer complementary to the ligated adaptor, such that DNA polymerization can proceed across the ChIP DNA. dsDNA is then synthesized from each single-stranded DNA by primer extension using phi29 DNA polymerase (or equivalent).

Following primer extension, the dsDNA molecule undergoes an A-tailing reaction to generate a single-stranded A overhang on the 3′ end of the dsDNA molecule. Following A-tailing, at least one adaptor nucleic acid molecule is ligated to the dsDNA molecule in a second adaptor ligation step. In one embodiment, the second adaptor ligation step includes annealing a splint adaptor to the nucleic acid fragment using a splint adaptor ligation method. Any appropriate method of ligating a splint adaptor may be used in the method of the invention. In one embodiment, the method comprises the use of T4 DNA ligase to anneal a splint adaptor to the eluted nucleic acid molecules.

Following ligation of a second adaptor molecule, the nucleic acid molecule is denatured to generate a ssDNA molecule. The resulting eluted single-stranded nucleic acid molecule is contacted with at least one primer having a sequence complementary to a sequence of at least one ligated adaptor, such that DNA polymerization can proceed across the ChIP nucleic acid molecule. This nucleic acid molecule can then be amplified by PCR or LM-PCR.

Alternatively, in one embodiment, a second adaptor molecule is ligated to the exonuclease digested nucleic acid molecule prior to elution. In such an embodiment, following exonuclease digestion, a second adaptor nucleic acid molecule is ligated to the crosslinked nucleic acid molecule using a single stranded nucleic acid molecule ligation method. Any appropriate method of ligating a single stranded nucleic acid molecule may be used in the method of the invention. In one embodiment, the method comprises the use of T4 DNA ligase to anneal a single-stranded adapter to the 5′ ends of the digested crosslinked nucleic acid molecules.

In one embodiment, the second adaptor comprises a pool of adaptors which each contain at least 2, at least 3, at least 4, at least 5, at least 6, at least 7, at least 8, at least 9, or at least 10 random nucleotides at the 3′ terminus. In one embodiment, the first adaptor comprises one of a pool of adaptors having a nucleotide sequence as set forth in SEQ ID NO:7, wherein each of the indicated “N” nucleotides can be any nucleotide.

Following ligation of a second adaptor, the protein-nucleic acid molecule complex is eluted from the resin, by reversing the crosslinks.

The ligated nucleic acid molecule is then amplified by polymerase chain reaction (PCR). In one embodiment, additional adaptor sequences are added by PCR. Since different efficiencies of amplification might occur with each fragment (e.g. small fragments amplify better than larger ones), the number of cycles should be kept to a minimum. Regardless, statistical methods are known in the art and can be used in the methods of the invention to detect and correct for potential PCR biases (e.g. number of unique adaptor/ChIP-DNA borders).

Modified ChIP-Exo

In various embodiments, one or more of the sequencing adaptors are annealed to the target nucleic acid molecule through a single-stranded ligation or splint ligation method. In some embodiments, a combination of single-stranded ligation and splint ligation can be used in a modified ChIP-exo method for generating a library of tagged nucleic acid molecule fragments for use as next-generation sequencing or amplification templates.

In one embodiment, the modified ChIP-exo method of the invention comprises the steps of: crosslinking a protein of interest to a nucleic acid molecule, fragmenting the nucleic acid molecule, immunoprecipitating the protein of interest, contacting the crosslinked nucleic acid molecule fragments with at least one 5′→3′ exonuclease to generate a single-stranded nucleic acid region on the crosslinked nucleic acid molecule fragments, ligating a first adaptor molecule to the single-stranded nucleic acid region, reversing the crosslinks to elute the nucleic acid molecule, ligating a second adaptor molecule to the eluted nucleic acid molecule, performing PCR amplification of the ligated molecule, and sequencing the PCR amplified products.

In one embodiment, the resulting nucleic acid molecule sample is used for high-throughput sequencing, using, for example, the Illumina/Solexa GAII, AB SOLiD system, Ion Torrent PGM, Ion Proton, Illumina MiSeq, Illumina HiSeq 2000 or 2500 and the like.

ChIP-Exo 4.0

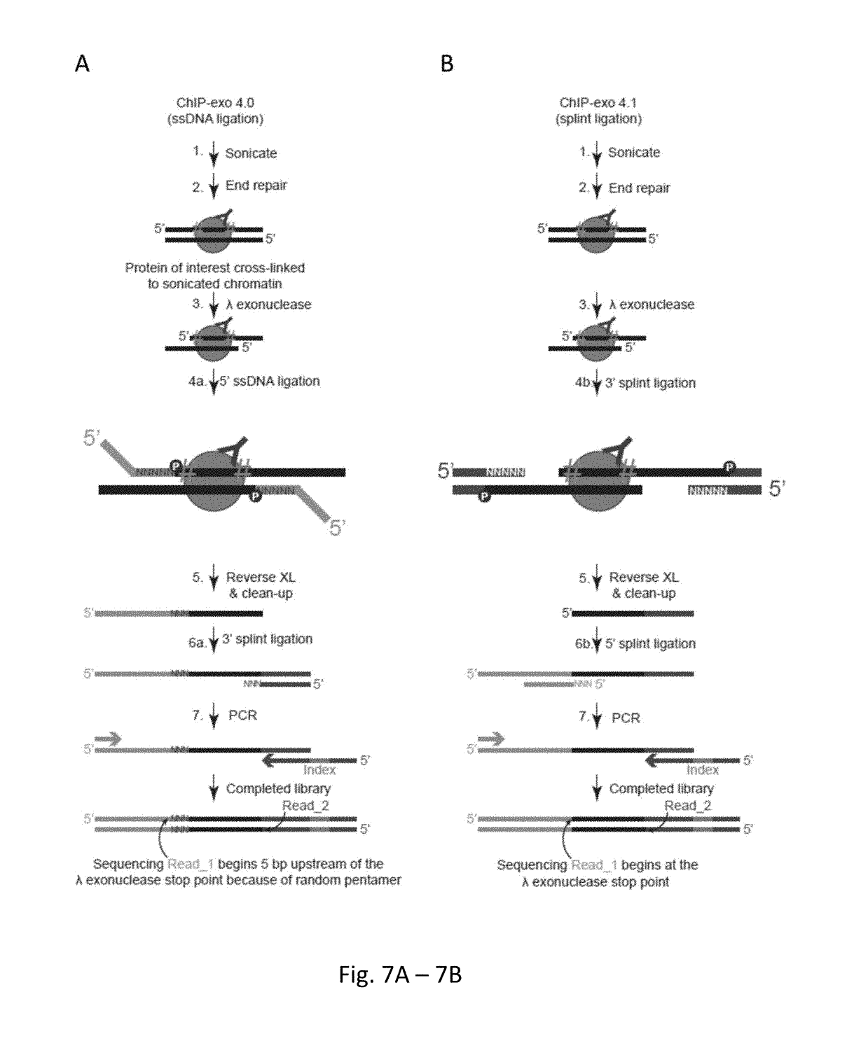

In one embodiment, the method of ligating a first adaptor molecule to the single-stranded nucleic acid region comprises a 5′ ssDNA ligation method. An exemplary procedure for this method, which is referred to as ChIP-exo 4.0, is depicted in FIG. 7A. In the ChIP-exo 4.0 assay, cells are crosslinked with formaldehyde and lysed. The crosslinked nucleic acid molecule is then fragmented and end repaired to generate blunt ends on the crosslinked nucleic acid molecule fragments prior to immunoprecipitation. The protein of interest is then immunoprecipitated. Following immunoprecipitation the sample then remains on the resin during the digestion and first adaptor ligation steps. In one embodiment, the digestion step comprises contacting the resin-bound crosslinked nucleic acid molecule fragments with a 5′→3′ exodeoxyribonuclease, to digest one nucleic acid molecule strand up to the bound protein.

After lambda exonuclease digestion, at least one adaptor nucleic acid molecule is ligated to the crosslinked nucleic acid molecule in a first adaptor ligation step. In one embodiment, the first adaptor ligation step includes ligating an adaptor to the immunoprecipitated nucleic acid fragment using a single stranded nucleic acid molecule ligation method. Any appropriate method of ligating a single stranded nucleic acid molecule may be used in the method of the invention. In one embodiment, the method comprises the use of T4 DNA ligase to anneal a single-stranded adapter to the 5′ ends of the digested crosslinked nucleic acid molecules.

In one embodiment, the first adaptor comprises a pool of adaptors which each contain at least 2, at least 3, at least 4, at least 5, at least 6, at least 7, at least 8, at least 9, or at least 10 random nucleotides at the 3′ terminus. In one embodiment, the first adaptor comprises one of a pool of adaptors having a nucleotide sequence as set forth in SEQ ID NO:7, wherein each of the indicated “N” nucleotides can be any nucleotide.

Following ligation of a first adaptor, the protein-nucleic acid molecule complex is eluted from the resin, by reversing the crosslinks.

After elution, at least one adaptor nucleic acid molecule is ligated to the crosslinked nucleic acid molecule in a second adaptor ligation step. In one embodiment, the second adaptor ligation step includes ligating a splint adaptor to the nucleic acid fragment using a splint adaptor ligation method. Any appropriate method of ligating a splint adaptor may be used in the method of the invention. In one embodiment, the method comprises the use of T4 DNA ligase to anneal a splint adaptor to the 3′ ends of the eluted nucleic acid molecules.

In one embodiment, the splint adaptor comprises one of a pool of splint adaptors having a dsDNA portion and a single-stranded 3′ overhang which each contain at least 2, at least 3, at least 4, at least 5, at least 6, at least 7, at least 8, at least 9, or at least 10 random nucleotides at the 3′ terminus of the single-stranded 3′ overhang. In one embodiment, the splint adaptor comprises one of a pool of duplexes formed from basepairing of nucleic acid molecules having a sequence as set forth in SEQ ID NO:6 with a pool of nucleic acid molecules having a nucleotide sequence as set forth in SEQ ID NO:5, wherein each of the indicated “N” nucleotides of SEQ ID NO:5 can be any nucleotide.

Following splint ligation, the nucleic acid molecule is denatured to generate ssDNA molecule. The resulting eluted single-stranded nucleic acid molecule is contacted with at least one primer having a sequence complementary to sequence of a ligated adaptor, such that DNA polymerization can proceed across the ChIP nucleic acid molecule. This nucleic acid molecule can then be amplified by PCR or LM-PCR.

ChIP-Exo 4.1

In one embodiment, both the method of ligating the first adaptor molecule to the single-stranded nucleic acid region and the method of ligating the second adaptor molecule comprises a splint ligation method. An exemplary procedure for this method, which is referred to as ChIP-exo 4.1, is depicted in FIG. 7B. In the ChIP-exo 4.1 assay, cells are crosslinked with formaldehyde and lysed. The crosslinked nucleic acid molecule is then fragmented. The protein of interest is then immunoprecipitated. Following immunoprecipitation the sample then remains on the resin during the digestion and first adaptor ligation steps. The nucleic acid molecule fragments are end repaired to generating blunt dsDNA ends on the crosslinked nucleic acid molecule fragments prior to exonuclease digestion. In one embodiment, the digestion step comprises contacting the resin-bound crosslinked nucleic acid molecule fragments with a 5′→3′ exodeoxyribonuclease, to digest one nucleic acid molecule strand up to the bound protein.

After lambda exonuclease digestion, at least one adaptor nucleic acid molecule is ligated to the crosslinked nucleic acid molecule in a first adaptor ligation step. In one embodiment, the first adaptor ligation step includes ligating an adaptor molecule to the immunoprecipitated nucleic acid fragment using a splint ligation method. Any appropriate method of ligating a splint adaptor may be used in the method of the invention. In one embodiment, the method comprises the use of T4 DNA ligase to anneal a splint adaptor to the 3′ ends of the eluted nucleic acid molecules.

In one embodiment, the splint adaptor comprises one of a pool of splint adaptors having a dsDNA portion and a single-stranded 3′ overhang which each contain at least 2, at least 3, at least 4, at least 5, at least 6, at least 7, at least 8, at least 9, or at least 10 random nucleotides at the 3′ terminus of the single-stranded 3′ overhang. In one embodiment, the splint adaptor comprises one of a pool of duplexes formed from basepairing of nucleic acid molecules having a sequence as set forth in SEQ ID NO:6 with a pool of nucleic acid molecules having a nucleotide sequence as set forth in SEQ ID NO:5, wherein each of the indicated “N” nucleotides of SEQ ID NO:5 can be any nucleotide.

Following ligation of a first adaptor, the protein-nucleic acid molecule complex is eluted from the resin, by reversing the crosslinks. After elution, at least one adaptor nucleic acid molecule is ligated to the crosslinked nucleic acid molecule in a second adaptor ligation step. In one embodiment, the second adaptor ligation step includes annealing a splint adaptor to the nucleic acid fragment using a splint adaptor ligation method. Any appropriate method of ligating a splint adaptor may be used in the method of the invention. In one embodiment, the method comprises the use of T4 DNA ligase to anneal a splint adaptor to the 5′ ends of the eluted nucleic acid molecules.

In one embodiment, the splint adaptor comprises one of a pool of splint adaptors having a dsDNA portion and a single-stranded 5′ overhang which each contain at least 2, at least 3, at least 4, at least 5, at least 6, at least 7, at least 8, at least 9, or at least 10 random nucleotides at the 5′ terminus of the single-stranded 5′ overhang. In one embodiment, the splint adaptor comprises one of a pool of duplexes formed from basepairing of nucleic acid molecules having a sequence as set forth in SEQ ID NO:12 with a pool of nucleic acid molecules having a nucleotide sequence as set forth in SEQ ID NO:14, wherein each of the indicated “N” nucleotides of SEQ ID NO:14 represents any nucleotide.

Following splint ligation, the nucleic acid molecule is denatured to generate a ssDNA molecule. The resulting eluted single-stranded nucleic acid molecule is contacted with at least one primer having a sequence complementary to a sequence of at least one ligated adaptor, such that DNA polymerization can proceed across the ChIP nucleic acid molecule. This nucleic acid molecule can then be amplified by PCR or LM-PCR.

ChIP-Exo 5.0

In one embodiment, the modified ChIP-exo method of the invention comprises the steps of: crosslinking a protein of interest to a nucleic acid molecule, fragmenting the nucleic acid molecule, immunoprecipitating the protein of interest, A-tailing the crosslinked nucleic acid molecules, ligating a first adaptor molecule to the nucleic acid molecule in a reaction that includes polynucleotide kinase, contacting the ligated nucleic acid molecule fragments with at least one 5′→3′ exonuclease to generate a single-stranded nucleic acid region on the crosslinked nucleic acid molecule fragments, reversing the crosslinks to elute the nucleic acid molecule, ligating a second adaptor molecule to the eluted nucleic acid molecule, performing PCR amplification of the ligated molecule, and sequencing the PCR amplified products.

In one embodiment, the resulting nucleic acid molecule sample is used for high-throughput sequencing, using, for example, the Illumina/Solexa GAII, AB SOLiD system, Ion Torrent PGM, Ion Proton, Illumina MiSeq, Illumina HiSeq 2000 or 2500 and the like.

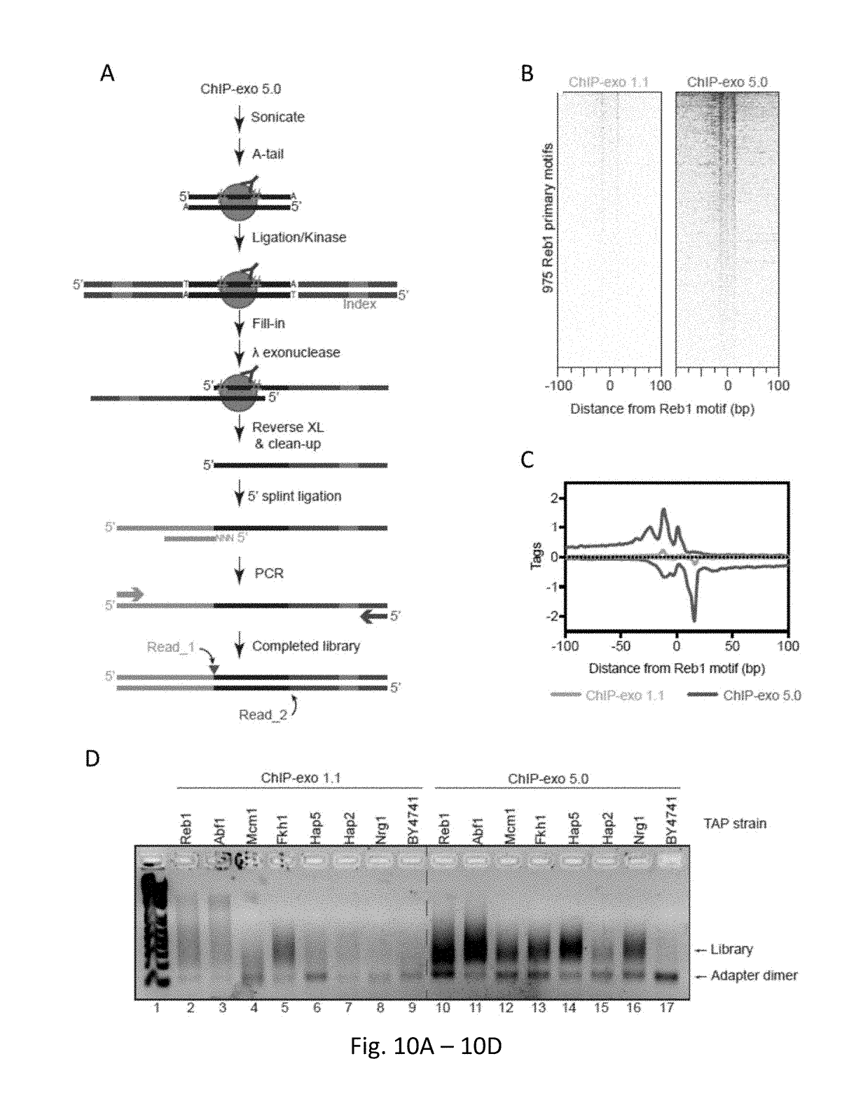

An exemplary procedure for this method, which is referred to as ChIP-exo 5.0, is depicted in FIG. 10A. In the ChIP-exo 5.0 assay, cells are crosslinked with formaldehyde and lysed. The crosslinked nucleic acid molecule is then fragmented. The protein of interest is then immunoprecipitated. Following immunoprecipitation the sample then remains on the resin during the first adaptor ligation and digestion steps. Prior to the first adaptor ligation step, the method comprises A-tailing of the crosslinked nucleic acid molecules. The first adaptor ligation is then performed in combination with a kinase reaction wherein the A-tailed nucleic acid molecules are contacted with a single reaction mixture comprising a first adaptor to be ligated, a ligase, and a kinase. In one embodiment, the kinase is a T4 Polynucleotide Kinase (T4 PNK). In one embodiment, the ligase is T4 DNA ligase.

In one embodiment, the adaptor comprises a dsDNA portion and a single-stranded 5′ overhang which each contain a barcode sequence internally in the single-stranded 5′ overhang. In one embodiment, the adaptor comprises a duplex formed from basepairing of a nucleic acid molecule having a sequence as set forth in SEQ ID NO:9 with a nucleic acid molecule having a nucleotide sequence as set forth in SEQ ID NO:8, wherein the indicated “X” nucleotides of SEQ ID NO:8 indicate any length or sequence of barcode nucleotides.

In one embodiment, the uniqueness of mapped 5′ ends of paired-end reads serve as the functional equivalent of random barcodes. Two reads that have identical Read1 5′ ends and identical Read2 5′ ends are deemed to be PCR duplicates, and are discarded.

In one embodiment, the digestion step comprises contacting the resin-bound crosslinked nucleic acid molecule fragments with a 5′→3′ exodeoxyribonuclease, to digest one nucleic acid molecule strand up to the bound protein. After lambda exonuclease digestion, the protein-nucleic acid molecule complex is eluted from the resin by reversing the crosslinks.

After elution, at least one adaptor nucleic acid molecule is ligated to the crosslinked nucleic acid molecule in a second adaptor ligation step. In one embodiment, the second adaptor ligation step includes annealing a splint adaptor to the nucleic acid fragment using a splint adaptor ligation method. Any appropriate method of ligating a splint adaptor may be used in the method of the invention. In one embodiment, the method comprises the use of T4 DNA ligase to anneal a splint adaptor to the 5′ ends of the eluted nucleic acid molecules.

In one embodiment, the splint adaptor comprises one of a pool of splint adaptors having a dsDNA portion and a single-stranded 5′ overhang which each contain at least 2, at least 3, at least 4, at least 5, at least 6, at least 7, at least 8, at least 9, or at least 10 random nucleotides at the 5′ terminus of the single-stranded 5′ overhang. In one embodiment, the splint adaptor comprises one of a pool of duplexes formed from basepairing of nucleic acid molecules having a sequence as set forth in SEQ ID NO:12 with a pool of nucleic acid molecules having a nucleotide sequence as set forth in SEQ ID NO:14, wherein each of the indicated “N” nucleotides of SEQ ID NO:14 represents any nucleotide.

Following splint ligation, the nucleic acid molecule is denatured to generate a ssDNA molecule. The resulting eluted single-stranded nucleic acid molecule is contacted with at least one primer having a sequence complementary to a sequence of at least one ligated adaptor, such that DNA polymerization can proceed across the ChIP nucleic acid molecule. This nucleic acid molecule can then be amplified by PCR or LM-PCR.

Modified ChIP-Seq

In one embodiment, the method of the invention comprises a modified ChIP-seq method comprising the steps of: crosslinking a protein of interest to a nucleic acid molecule, fragmenting the nucleic acid molecule, immunoprecipitating the protein of interest, ligating a first adaptor molecule and a second adaptor molecule to the nucleic acid molecule in a single reaction, reversing the crosslinks to elute the nucleic acid molecule, performing PCR amplification of the ligated molecule, and sequencing the PCR amplified products.

In one embodiment, the resulting nucleic acid molecule sample is used for high-throughput sequencing, using, for example, the Illumina/Solexa GAII, AB SOLiD system, Ion Torrent PGM, Ion Proton, Illumina MiSeq, Illumina HiSeq 2000 or 2500 and the like.

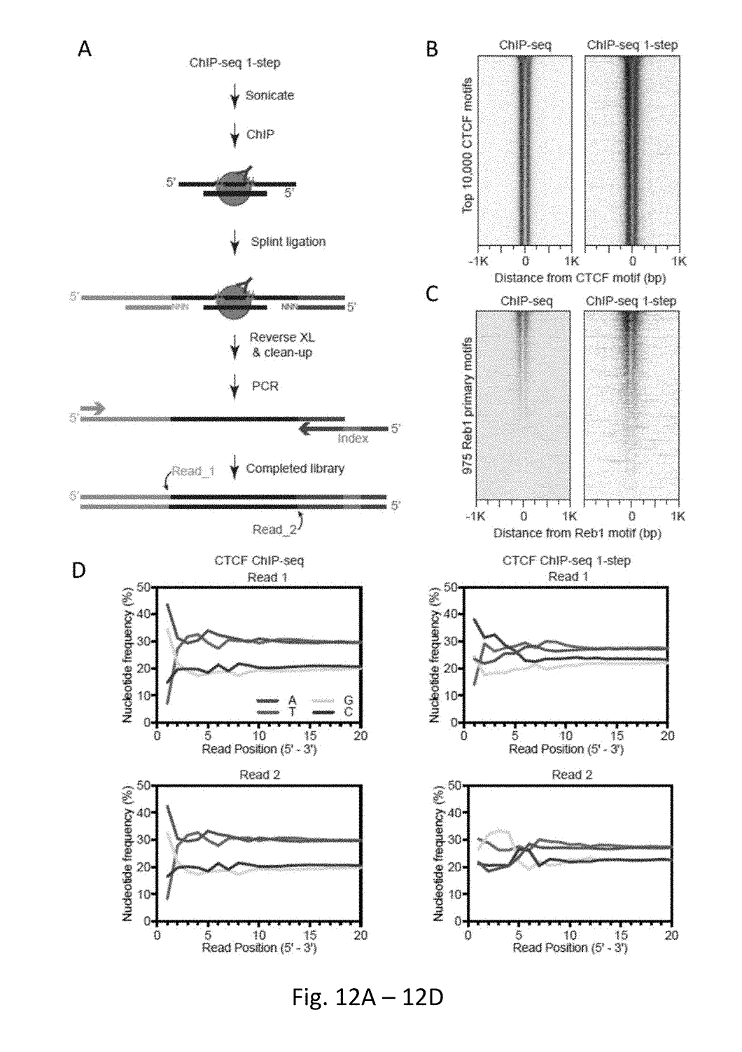

An exemplary procedure for this method, which is referred to as ChIP-seq 1-step, is depicted in FIG. 12A. In the ChIP-seq 1-step assay, cells are crosslinked with formaldehyde and lysed. The crosslinked nucleic acid molecule is then fragmented. The protein of interest is then immunoprecipitated.

Following immunoprecipitation the sample then remains on the resin during the adaptor ligation step. In one embodiment, the adaptor ligation comprises dual ligation of two splint adaptors.

In one embodiment, a first splint adaptor comprises one of a pool of adaptors, each having a dsDNA portion and a single-stranded 5′ overhang, which each contain at least 2, at least 3, at least 4, at least 5, at least 6, at least 7, at least 8, at least 9, or at least 10 random nucleotides at the 5′ terminus of the single-stranded 5′ overhang. In one embodiment, the first splint adaptor comprises one of a pool of duplexes formed from basepairing of nucleic acid molecules having a sequence as set forth in SEQ ID NO:12 with a pool of nucleic acid molecules having a nucleotide sequence as set forth in SEQ ID NO:14, wherein each of the indicated “N” nucleotides of SEQ ID NO:14 represents any nucleotide.

In one embodiment, a second splint adaptor comprises one of a pool of adaptors, each having a dsDNA portion and a single-stranded 3′ overhang, which each contain at least 2, at least 3, at least 4, at least 5, at least 6, at least 7, at least 8, at least 9, or at least 10 random nucleotides at the 3′ terminus of the single-stranded 3′ overhang. In one embodiment, the second splint adaptor comprises one of a pool of duplexes formed from basepairing of nucleic acid molecules having a sequence as set forth in SEQ ID NO:6 with a pool of nucleic acid molecules having a nucleotide sequence as set forth in SEQ ID NO:5, wherein each of the indicated “N” nucleotides of SEQ ID NO:5 represents any nucleotide.

In one embodiment, the first adaptor molecule and the second adaptor molecule are ligated to the fragmented crosslinked nucleic acid molecule in a single reaction. In one embodiment, the ligation is performed using T4 DNA ligase.

After the ligation step, the protein-nucleic acid molecule complex is eluted from the resin by reversing the crosslinks. Following elution, the nucleic acid molecule is denatured to generate a ssDNA molecule. The resulting eluted single-stranded nucleic acid molecule is contacted with at least one primer having a sequence complementary to a sequence of at least one ligated adaptor, such that DNA polymerization can proceed across the ChIP nucleic acid molecule. This nucleic acid molecule can then be amplified by PCR or LM-PCR.

### Biological Sample

The biological sample can be any sample from which genomic nucleic acid can be obtained. In one embodiment, the target DNA represents a sample of genomic DNA isolated from a cell or a subject. The biological sample(s) can be prepared using methodologies well known in the art such as by obtaining a specimen from an individual and, if necessary, disrupting any cells contained thereby to release genomic nucleic acids.

Biological samples which can be tested by the methods of the present invention described herein include human cells, tissues and body fluids such as whole blood, serum, plasma, cerebrospinal fluid, sputum, bronchial washing, bronchial aspirates, urine, lymph fluids and various external secretions of the respiratory, intestinal and genitourinary tracts, tears, saliva, milk, white blood cells, myelomas, buccal cells, cervicovaginal cells, epithelial cells from urine, fetal cells, or any cells present in tissue obtained by biopsy and the like; biological fluids such as cell culture supernatants; tissue specimens which may be fixed; and cell specimens which may be fixed.

This DNA may be obtained from any cell source, tissue source, or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen and tissue exudates at a site of infection or inflammation. DNA is extracted from the cell source, tissue source, or body fluid using any of the numerous methods that are standard in the art. It will be understood that the particular method used to extract DNA will depend on the nature of the source.

In one embodiment, multiple samples are amplified individually using the method of the invention and pooled together prior to sequencing using a Next Gen Sequencing platform. In one embodiment, multiple samples may be from the same type of biological sample (e.g. all FFPE samples). In one embodiment, multiple samples may be from different types of biological samples.

### Nucleic Acid Samples and Preparation

As contemplated herein, the present invention may be used in the analysis of any nucleic acid sample for which next generation sequencing may be applied. For example, the nucleic acid can be from a cultured cell or cells or a patient cell or tissue or bodily fluid sample. The nucleic acid may be isolated using methods generally known to those of skill in the art, including, methods which preserve protein-DNA interactions and methods which are readily immobilized or immunoprecipitated.

The nucleic acid may be prepared (e.g., library preparation) for massively parallel sequencing in any manner as would be understood by those having ordinary skill in the art. While there are many variations of library preparation, the purpose is to construct nucleic acid fragments of a suitable size for a sequencing instrument and to modify the ends of the sample nucleic acid to work with the chemistry of a selected sequencing process. Depending on application, nucleic acid fragments may be generated having a length of about 25 to about 1000 bases. It should be appreciated that the present invention can accommodate any nucleic acid fragment size range that can be read by a sequencer. This can be achieved by selecting primers such that the resulting PCR product is within the desired range specific for the sequencer and sequencing method desired. For example, in various embodiments a desired PCR fragment size, including barcode and adaptor regions is about 100, 150, 200, 250, 300, 350, 400, 450 or about 500 bp. Both the 5′ and 3′ ends of the PCR products comprise nucleic acid adapters. In various embodiments, these adapters have multiple roles, such as allowing attachment of the specimen strands to a substrate (bead or flow cell) and having a nucleic acid sequence that can be used to initiate the sequencing reaction through hybridization to a sequencing primer. Further, in some embodiments, the PCR products also contain unique sequences (bar-coding) that allow for identification of individual samples in a multiplexed run. The key component of this attachment process is that each individual PCR product is attached to a bead or location on a slide or flow cell. This single PCR fragment can then be further amplified to generate hundreds of identical copies of itself in a clustered region on the bead, flow cell or slide location. These clusters of identical DNA form the product that is sequenced by any one of several next generation sequencing technologies.

The samples can be sequenced using any massively parallel sequencing platform. Non-limiting examples of sequencers include Illumina/Solexa GAII, AB SOLiD system, Ion Torrent PGM, Ion Proton, Illumina MiSeq, Illumina HiSeq 2000 or 2500 and the like.

### PCR Primers

In various embodiments, the assay comprises a combination of at least one forward and at least one reverse PCR primer. In some embodiments, a forward primer of the invention comprises at least a region complementary to a sequence of an adaptor molecule that has been ligated to a target nucleic acid molecule. In some embodiments, a reverse primer of the invention comprises at least one of a region complementary to a sequence of an adaptor molecule that has been ligated to a target nucleic acid molecule, a sample barcode region, and a sequencing adaptor region. The sequencing adaptor region allows for hybridization to a NGS-based sequencing platform, such as a bead or flow cell. In one embodiment, a sequencing adaptor region comprises a sequence specific for use in an Ion Torrent sequencing system. In one embodiment, a sequencing adaptor region comprises a sequence specific for use in an Illumina sequencing system.

In some embodiments, the forward PCR primer comprises the sequence of

SEQ ID NO:15. In some embodiments, the reverse PCR primer comprises the sequence of SEQ ID NO:17. In some embodiments, in the reverse PCR primer, the sequencing adaptor region is located 5′ to the sample barcode region which is 5′ to a region complementary to a sequence of an adaptor molecule that has been ligated to a target nucleic acid molecule.

### Methods of Identification of Binding Sites

As contemplated herein, the present invention includes methods of analyzing Next Gen Sequencing data. Generally, sequence reads are aligned, or mapped, to a reference sequence using, for example, available commercial software or open source freeware (e.g., nucleotide and quality data input, mapped reads output). This may include preparation of read data for processing using format conversion tools and optional quality and artifact removal filters before passing the read data to an alignment tool. Next, variants are called (e.g., summarized data input, variant calls output) and interpreted (e.g., variant calls input, genotype information output).

Standard approaches to mapping and analysis of this type of massively parallel sequence data are applicable to the invention described herein. In some embodiments, an analytical pipeline may detect the binding sites of a protein of interest, as outlined in the method below. First, raw read data, which may include sequence and quality information from the sequencing hardware, is received and entered into the system. The data is optionally prefiltered, for example, one read at a time or in parallel, to remove data that is too low in quality, typically by end trimming or rejection. For a multiplexed sequencing reaction, the raw reads are sorted according to the barcode region to group reads from each individual sample. The reads are then trimmed to remove barcode and adaptor sequences.

The remaining data is then aligned using a set of reference sequences. Read data can be mapped to reference sequences using any mapping software, and using appropriate alignment and sensitivity settings suitable for the goal of the project. Mapped reads may optionally be postfiltered to remove low quality or uncertain mappings. The total numbers of aligned reads can be determined using any appropriate method including, but not limited to, SAMtools, a PERL script, a PYTHON script, and a sequencing analysis pipeline.

In various embodiments, at least 1000, at least 2000, at least 3000, at least 4000, at least 5000, at least 10,000, at least 50,000, at least 100,000, at least 500,000 or more than 500,000 sequencing reads are determined to be ‘high quality’ after passing quality filters. In one embodiment, ‘high quality’ sequencing reads are aligned to one or more reference sequences.

### Kits

In one embodiment, the invention provides a kit for use in the modified ChIP-seq or modified ChIP-exo methods of the invention. The kit comprises one or more of: (a) reagents to wash chromatin; (b) reagents for carrying out end repair; (c) reagents for carrying out dA tailing; (d) an adaptor; (e) reagents for ligating the adaptor to the chromatin; (0 reagents for filling in 5′ overhang in the chromatin caused by the adaptor; (g) reagents for end trimming; (h) reagents for carrying out 5′-3′ double-stranded-specific exonuclease digestion; (i) reagents for carrying out 5′-3′ single-stranded-specific exonuclease digestion; and (j) reagents for carrying out PCR amplification of the polynucleotide sequence.

Reagents to wash chromatin include, but are not limited to, FA Lysis Buffer, NaCl Buffer (50 mM HEPES-KOH, pH 7.5, 500 mM NaCl, 2 mM EDTA, 1% Triton-X 100, 0.1% sodium deoxycholate), Tris-EDTA buffer, Triton X-100, mixed micelle buffer, buffer 500, LiCl/detergent buffer (100 mM Tris-HCl, pH 8.0, 500 mM LiCl, 1% NP-40, 1% sodium deoxycholate), and Tris-HCl.

Reagents for carrying out end repair include, but are not limited to, DNA polymerase I, large fragment, T4 DNA polymerase, T4 polynucleotide kinase, dNTPs, and T4 ligase buffer.

Reagents for carrying out dA tailing include, but are not limited to, Klenow fragment (3′-5′, exo minus), ATP, and NEBuffer 2.

Reagents for ligating the adaptor to the chromatin include, but are not limited to, T4 DNA ligase, adaptors (e.g., as set forth in Table 1), and T4 DNA Ligase Buffer.

Reagents for filling in 5′ overhangs in the chromatin caused by the adaptor include, but are not limited to, Klenow fragment (3′-5′ exo”), dNTPs, and NEBuffer 2.

Reagents for end trimming include, but are not limited to, T4 DNA polymerase, dNTPs, and T4 ligase buffer.

Reagents for carrying out 5′-3′ double-stranded-specific exonuclease digestion include, but are not limited to, lambda exonuclease, DMSO, Triton X-100, and lambda exonuclease reaction buffer.

Reagents for carrying out 5′-3′ single-stranded-specific exonuclease digestion include, but are not limited to, Rech exonuclease, DMSO, Triton X-100, and NEBuffer 2.

Reagents for carrying out PCR amplification of a polynucleotide sequence include, but are not limited to, polymerase buffer, dNTPs, universal and barcode primers, a DNA polymerase, water, and DNA. Exemplary buffer recipes and components are well known to those of skill in the art.

Any kit of the invention may also include suitable instructional material, storage containers, e.g., ampules, vials, tubes, etc., for each reagent disclosed herein, an reagents used as controls, e.g., a positive control nucleic acid sequence or positive control antibody). The reagents may be present in the kits in any convenient form, such as, e.g., in a solution or in a powder form. The kits may further include a packaging container, optionally having one or more partitions for housing the various reagents.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

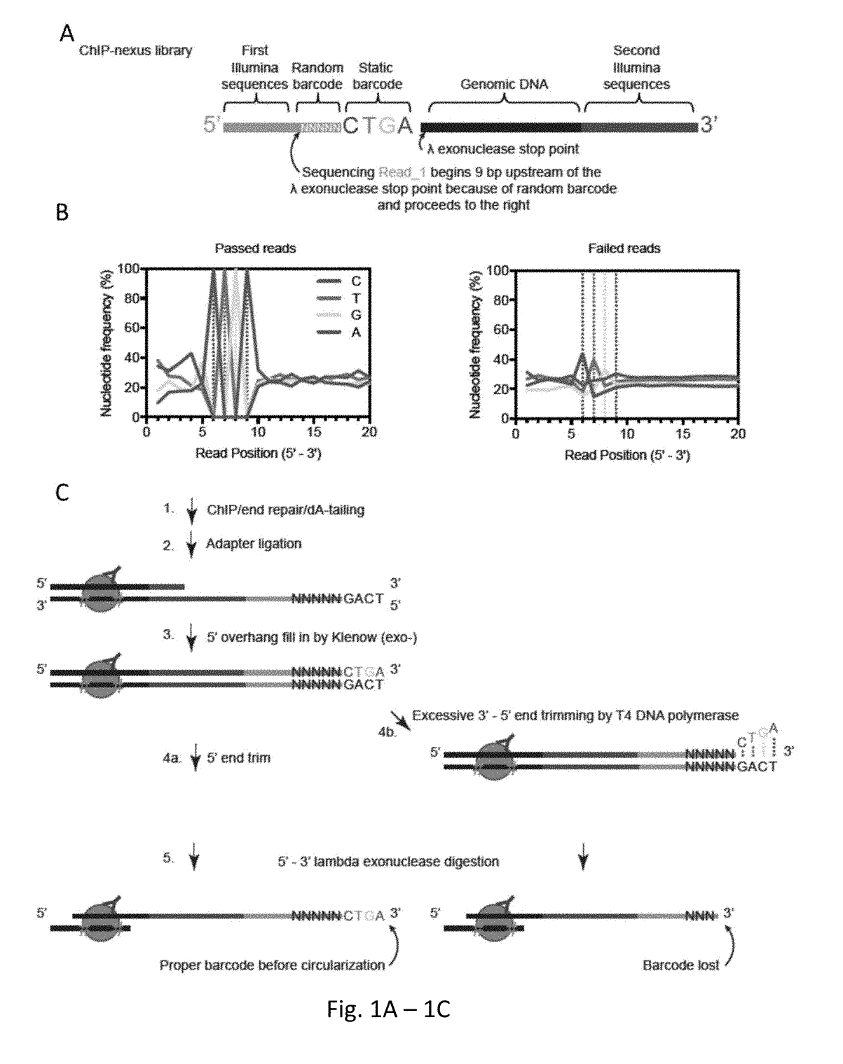

ChIP-seq was developed as a powerful method for determining chromatin-bound and transcription factor-bound regions of the genome (Albert et al., (2007) Nature, 446:572-576; Johnson et al., (2007) Science, 316:1497-1502). ChIP-exo 1.0 built upon that utility by taking advantage of factor-specific cross-linking patterns within each DNA binding event to achieve the following: 1) improve signal-to-noise detection, thereby providing more a comprehensive set of bound locations, 2) elucidate the positional organization of proteins within a complex, and 3) detect alternative binding modes. Technical difficulty and sequencing platform restriction of the original assay may have limited broader adoption. Version 1.1 brought ChIP-exo to the Illumina platform. Version 2.0 (ChIP-nexus) provided some simplification by eliminating two enzymatic steps, but required multiple ligations steps and an extra restriction endonuclease step. Version 2.0 also resulted in some data loss, possibly due to unintended enzymatic loss of barcode information (FIG. 1).

To simplify the assay, a systematic effort was undertaken. This included a reduction in the number of enzymatic steps and alternative strategies for adapter ligation. This simplification process is also applicable to ChIP-seq library construction, in essence resulting in a one-step library construction. The practical benefits of improved library construction include reduced costs, reduced processing time, and increased yield. Multiple alternative versions were developed because, in addition to each producing the expected resolution, each has particular trade-offs of advantages and limitations that may make each method more suitable for particular applications.

ChIP-exo 3.0, presented here, takes advantage of one-step adapter attachment afforded by Tn5 tagmentation. This version is technically simpler than all prior versions of ChIP-exo and retains ultra-high resolution. However, it produces “shouldering”, which is essentially a signal distribution pattern that is equivalent to ChIP-seq and ChIPmentation. Thus, version 3.0 may be useful where assay simplicity is paramount and a blend of ChIP-exo and ChIP-seq signal patterning is acceptable.

ChIP-exo 4.0/4.1 was developed to streamline library construction in a way that avoided bias. Both 4.0 and 4.1 involve ligating the first adapter after lambda exonuclease digestion, with version 4.0 ligating a ssDNA adapter (corresponding to Read_1) to the resected ChIP-exo DNA. Version 4.1 uses a splint in ligating to the non-resected end (Corresponding to Read_2). Both use a splint in the second ligation step. These versions are both technically quite simple and lack the bias of other methods. However, both displayed shouldering as seen in version 3.0. Thus, versions 4.0/4.1 are technically the simplest of all versions, and may be the method of choice where ChIP-exo patterning is desired, but where some level of lower-resolution ChIP-seq quality signal can be tolerated.

ChIP-exo 5.0 was developed to alleviate shouldering. In total, thirteen enzymatic steps were reduced to five. ChIP-exo 5.0 is the most suitable assay to maximize signal concentration from ChIP-exo patterning. Without being bound by a particular theory, it is hypothesized that the initial A-tailing and adapter ligation may select for ChIP DNA molecules that are subsequently digestible by lambda exonuclease, thereby eliminating shoulders.

Finally, the advantages of ChIP-exo version 4.1 were incorporated into ChIP-seq to create a highly-streamlined ChIP-seq assay that includes library construction in a single step, called ChIP-seq 1-step. Essentially, ChIP libraries are constructed by concurrent ssDNA and splint ligation of two adapters.

The value of ChIP-exo over ChIP-seq is the insight provided by exonuclease patterning, and the increased signal:noise that adds greater confidence to location calling. All versions of ChIP-exo in principle produce essentially the same lambda exonuclease pattern, so switching across any version of the assay, as might occur in an extended series of experiments, should have little impact on the qualitative conclusions drawn. Since all versions of ChIP-exo are a derivative of ChIP-seq, ChIP-exo data can be converted to ChIP-seq data. In fact, Read_2 from paired-end sequencing of ChIP-exo libraries is essentially a ChIP-seq signal. Therefore, even in the most highly refined ChIP-exo protocol, a pure ChIP-seq signal is separately produced and available for analysis. It (Read_2) was used to improve tag mappability. The newly developed ChIP-exo assays are performed in 8-well strip tubes using multichannel pipettors. This allows for significant scale-up. These refined ChIP-exo assays, particularly version 5.0, may be highly suitable for large genome-wide mapping projects (Consortium, (2012) Nature, 489:57-74).

The Materials and Methods used are now described

K562 Chromatin Preparation

Human chronic myelogenous leukemia cells (1(562, ATCC) were maintained between 1×10⁵ and 1×10⁶ cell/ml in DMEM media supplemented with 10% fetal bovine serum at 37° C. with 5% CO2. Cells were washed with PBS (8 mM Na₂HPO₄, 2 mM KH₂PO₄, 150 mM NaCl, and 2.7 mM KCl), then cross-linked with formaldehyde at a final concentration of 1% for 10 minutes at room temperature, and quenched with a final concentration of 125 mM glycine for 5 minutes. The supernatant was removed, and the cells were resuspended in 1 ml PBS to wash. Cells were aliquoted to contain 100 million cells, centrifuged, the supernatant was removed, and the pellet was flash frozen.

A 100 million cell aliquot (for use in multiple ChIPs) was lysed in 500 μl (10 mM Tris-HCl, pH 8.0, 10 mM NaCl, 0.5% NP40, and complete protease inhibitor (CPI, Roche)) by incubating on ice for 10 minutes. The lysate was microcentrifuged at 2,500 rpm for 5 minutes at 4° C. The supernatant was removed, the pellet resuspended in 1 ml (50 mM Tris-pH 8.0, 10 mM EDTA, 0.32% SDS, and CPI), and incubated on ice for 10 minutes to lyse the nuclei. The sample was diluted with 600 μl of immunoprecipitation dilution buffer (IP Dilution Buffer: 20 mM Tris-HCl, pH 8.0, 2 mM EDTA, 150 mM NaCl, 1% Triton X-100, and CPI) to a final concentration of (40 mM Tris-HCl, pH 8.0, 7 mM EDTA, 56 mM NaCl, 0.4% Triton-X 100, 0.2% SDS, and CPI), and sonicated with a Bioruptor (Diagenode) for 10 cycles with 30 second on/off intervals to obtain DNA fragments 100 to 500 bp in size.

Yeast Chromatin Preparation

TAP-tagged Saccharomyces cerevisiae strains were grown in 500 ml of yeast peptone dextrose (YPD) media to an OD600=0.8 at 25° C. Cells were cross-linked with formaldehyde at a final concentration of 1% for 15 minutes at room temperature, and quenched with a final concentration of 125 mM glycine for 5 minutes. Cells were collected by centrifugation, and washed in 1 ml of ST Buffer (10 mM Tris-HCl, pH 7.5, 100 mM NaCl) at 4° C. and split into two aliquots. The cells were pelleted again, the supernatant was removed, and the pellet was flash frozen.

A 250 ml culture aliquot was lysed in 750 μl of FA Lysis Buffer (50 mM Hepes-KOH, pH 7.5, 150 mM NaCl, 2 mM EDTA, 1% Triton, 0.1% sodium deoxycholate, and CPI) and 1 ml volume of 0.5 mm zirconia/silica beads by bead beating in a Mini-Beadbeater-96 machine (Biospec) for three cycles of 3 minutes on/5 minutes off cycles (Samples were kept on ice during the off cycle). The lysate was transferred to a new tube and microcentrifuged at maximum speed for 3 minutes at 4° C. to pellet the chromatin. The supernatant was discarded, and the pellet was resuspended in 750 μl of FA Lysis Buffer supplemented with 0.1% SDS and transferred to a 15 ml polystyrene conical tube. The sample was then sonicated in a Bioruptor (Diagenode) for 15 cycles with 30 second on/off intervals to obtain DNA fragments 100 to 500 bp in size.

Antibodies

Rabbit IgG (Sigma) conjugated to Dynabeads was used against TAP-tagged strains in which the TAP-tag containing Protein A was the target. Millipore 07-729 antibody was used against K562 samples targeting CTCF.

Tn5 Purification

A custom construct of Tn5 E54K E110K P242A L372P15 in a pET-45b(+) vector was ordered (Genescript) to express hyperactive Tn5 with an N-terminal His6-tag. BL21(DE3) competent E. coli cells (New England Biolabs) were transformed and a single colony was grown at 37° C. to an OD600 of 0.4 in 500 ml of LB+50 μg/ml ampicillin+30 μg/ml chloramphenicol. Cells were transferred to a 25° C. incubator and induced with 0.5 mM isopropyl-β-D-galactopyranoside for 4 hours. The cells were collected by centrifugation, washed once with ST Buffer, and the cell pellet was flash frozen in liquid nitrogen.

Tn5 was purified as previously described (Goryshin et al., (1998) J Biol Chem, 273:7367-7374), with few modifications. Cells were resuspended in 10 volumes (ml/g) of TEGX100 Buffer (20 mM Tris-HCl, pH 7.5, 100 mM NaCl, 1 mM EDTA, 10% glycerol, 0.1% Triton-X 100) containing CPI and 100 μM phenylmethylsulfonyl fluoride and lysed by incubation with lysozyme (Sigma; 1 mg/1 g of cell pellet) at room temperature for 30 minutes. The lysate was centrifuged at 20,000×g for 20 minutes at 4° C., and the supernatant was precipitated with 0.25% polyethyleneimine (Sigma) and centrifuged at 10,000×g for 15 minutes. The supernatant was then precipitated with 47% saturation ammonium sulfate (0.28 g/ml) over a 30 minutes incubation, and then centrifuged at 20,000×g for 15 minutes.

The pellet was then resuspended in 50 ml of Nickel Affinity Load Buffer (50 mM potassium phosphate, pH 7.4, 50 mM KCl, 20% glycerol) and loaded on a HisTrap HP column (GE Healthcare; 5 ml) at 1.5 ml/min equilibrated with the same buffer. The column was sequentially washed with Wash Buffer I (50 mM potassium phosphate, pH 7.4, 1 M KCl, 50 mM imidazole, 20% glycerol), Wash Buffer II (50 mM potassium phosphate, pH 7.4, 500 mM KCl, 50 mM imidazole, 20% glycerol), and then Tn5 was eluted with Nickel Affinity Elution Buffer (50 mM potassium phosphate, pH 7.4, 500 mM KCl, 500 mM imidazole, 20% glycerol) at 2 ml/min. The eluate was diluted to 300 mM KCl with Dilution Buffer (50 mM potassium phosphate, pH 7.4, 20% glycerol), and the final volume adjusted to 50 ml with TEGX300 Buffer (20 mM Tris-HCl, pH 7.5, 300 mM NaCl, 1 mM EDTA, 10% glycerol, 0.1% Triton-X 100).

Next, the sample was loaded on a HiTrap Heparin HP column (GE Healthcare; 1 ml) equilibrated with TEGX300 at 1 ml/min. After washing with 5 column volumes of buffer, a 10-ml linear (300 mM to 1.2 M) NaCl gradient was run to elute. Tn5 eluted from the column at approximately 600 mM NaCl. Fractions containing the main elution peak were combined (3.5 ml) and dialyzed overnight against TEGX300 Buffer containing 30% glycerol.

Chromatin Immunoprecipitation

A 50 ml culture-equivalent of yeast or 10 million cell-equivalent of K562 chromatin was diluted to 200 μl with IP Dilution Buffer and incubated overnight at 4° C. with the appropriate antibody. A 10 μl bed volume of IgG-Dynabeads was added to the yeast samples; and 3 μg of anti-CTCF antibody with a 10 μl slurry-equivalent of Protein A Mag Sepharose (GE Healthcare) was added to the K562 samples.

ChIP-Exo 1.1

ChIP-exo 1.1 was performed as previously described (Serandour et al., (2013) Genome Biol, 14:R147; Yen et al., (2013) Cell, 154:1246-1256; Rhee and Pugh (2012) Curr Protoc Mol Biol, Chapter 21, Unit 21 24). In brief, the following enzymatic steps were carried out with immunoprecipitated chromatin still on the resin with multiple salt washes between each step: T4 DNA polymerase end polishing, T4 polynucleotide kinase, Klenow fragment A-tailing, T4 DNA ligase-mediated Read_2 adapter ligation, phi29 DNA polymerase fill-in, second T4 polynucleotide kinase, lambda exonuclease digestion, and RecJ_{f} exonuclease digestion. Following overnight reverse cross-linking and Proteinase K treatment, the following steps were carried out in solution: phi29 primer extension, second Klenow fragment A-tailing, T4 DNA ligase-mediated Read_1 adapter ligation, and PCR.

ChIP-exo 3.0 (tagmentation-based version)

After immunoprecipitation, the following steps were carried out on the resin:

Transposase assembly: To allow Tn5 time to bind the adapter sequence, a 10× Transposase Mix was assembled with the following components and incubated for 30 minutes at room temperature: 12.5 μM Tn5, 50% glycerol, and 7.5 μM adapter (NexA2/ME comp). See Table 1 for oligonucleotide sequences used in this study.

TABLE 1
Oligonucleotides used in this study.
All sequences are written in the 5′-3′ direction
from left to right.

| | complement | | | | |
|---|---|---|---|---|---|
| oligo | oligo | | | length | |
| name | name | assay | step | (nt) | sequence |
| NexA2 | ME | ChIP- | tagmentation | 39 | /5Phos/AGACGTGTGCTCTTCCGATCAG |
| | comp | exo | | | ATGTGTATAAGAGACAG |
| | | 3.0 | | | (SEQ ID NO: 3) |
| ME | NexA2 | ChIP- | tagmentation | 19 | CTGTCTCTTATACACATCT |
| comp | | exo | | | (SEQ ID NO: 4) |
| | | 3.0 | | | |
| ExA2.1- | ExA2.1- | ChIP- | second | 25 | GACGTGTGCTCTTCTCTNNNNN |
| N5 | 20 | exo | adapter | | (SEQ ID NO: 5) |
| | | 4.0 | ligation | | |
| | | ChIP- | first | | |
| | | exo | adapter | | |
| | | 4.1 | ligation | | |
| ExA2.1- | ExA2.1- | ChIP- | second | 20 | /5Phos/AGATCGGAAGAGCACACGTC |
| 20 | N5 | exo | adapter | | (SEQ ID NO: 6) |
| | | 4.0 | ligation | | |
| | | ChIP- | first | | |
| | | exo | adapter | | |
| | | 4.1 | ligation | | |
| ExA1- | | ChIP- | first | 63 | AATGATACGGCGACCACCGAGATCTACAC |
| 58-N5 | | exo | adapter | | TCTTTCCCTACACGACGCTCT |
| | | 4.0 | ligation | | TCCGATCTNNNNN |
| | | | | | (SEQ ID NO: 7) |
| ExA2_ | ExA2B | ChIP- | first | 70 | /5Phos/CAAGCAGAAGACGGCATACGAG |
| iNN | | exo | adapter | | ATXXXXXXXXXXXXGTGACTGGAGTTCAG |
| | | 1.1 | ligation | | ACGTGTGCTCTTCCGATCT |
| | | ChIP- | | | (SEQ ID NO: 8) |
| | | exo | | | |
| | | 5.0 | | | |
| ExA2B | ExA2_ | ChIP- | first | 33 | GATCGGAAGAGCACACGTCTGAACTCCAG |
| | iNN | exo | adapter | | TCAC |
| | | 1.1 | ligation | | (SEQ ID NO: 9) |
| | | ChIP- | | | |
| | | exo | | | |
| | | 5.0 | | | |
| FX-15 | | ChIP- | primer | 15 | CTGGAGTTCAGACGT |
| | | exo | extension | | (SEQ ID NO: 10) |
| | | 1.1 | | | |
| ME | | ChIP- | primer | 19 | AGATGTGTATAAGAGACAG |
| sequence | | exo | extension | | (SEQ ID NO: 11) |
| | | 3.0 | | | |
| ExA1- | ExA1- | ChIP- | second | 58 | AATGATACGGCGACCACCGAGATCTACAC |
| 58 | 13 | exo | adapter | | TCTTTCCCTACACGACGCTCTTCCGATCT |
| | | 1.1 | ligation | | (SEQ ID NO: 12) |
| | | ChIP- | | | |
| | | exo | | | |
| | | 3.0 | | | |
| | ExA1- | ChIP- | second | | |
| | SSL_ | exo | adapter | | |
| | N5 | 4.1 | ligation | | |
| | | ChIP- | | | |
| | | exo | | | |
| | | 5.0 | | | |
| ExA1- | ExA1- | ChIP- | second | 13 | GATCGGAAGAGCG |
| 13 | 58 | exo | adapter | | (SEQ ID NO: 13) |
| | | 1.1 | ligation | | |
| | | ChIP- | | | |
| | | exo | | | |
| | | 3.0 | | | |
| ExA1- | ExA1- | ChIP- | second | 19 | NNNNNAGATCGGAAGAGCG |
| SSL_N5 | 58 | exo | adapter | | (SEQ ID NO: 14) |
| | | 4.1 | ligation | | |
| | | ChIP- | | | |
| | | exo | | | |
| | | 5.0 | | | |
| P1.3 | | ChIP- | PCR | 18 | AATGATACGGCGACCACC |
| | | exo | | | (SEQ ID NO: 15) |
| | | 1.1 | | | |
| | | ChIP- | | | |
| | | exo | | | |
| | | 3.0 | | | |
| | | ChIP- | | | |
| | | exo | | | |
| | | 4.0 | | | |
| | | ChIP- | | | |
| | | exo | | | |
| | | 4.1 | | | |
| | | ChIP- | | | |
| | | exo | | | |
| | | 5.0 | | | |
| NexA2- | | ChIP- | PCR | 69 | CAAGCAGAAGACGGCATACGAGATCXXXX |
| iNN | | exo | | | |
| | | 3.0 | | | XXXXXXXXTGACTGGAGTTCAGACGTGTG |
| | | ChIP- | | | CTCTTCCGATC |
| | | exo | | | (SEQ ID NO: 16) |
| | | 4.0 | | | |
| | | ChIP- | | | |
| | | exo | | | |
| | | 4.1 | | | |
| P2.1 | | ChIP- | PCR | 22 | CAAGCAGAAGACGGCATACGAG |
| | | exo | | | (SEQ ID NO: 17) |
| | | 1.1 | | | |
| | | ChIP- | | | |
| | | exo | | | |
| | | 5.0 | | | |

X index sequence that varies between samples for multiplexing
N random nucleotide sequence

ChIP wash: the resin was washed sequentially with FA Lysis Buffer, NaCl Buffer (50 mM HEPES-KOH, pH 7.5, 500 mM NaCl, 2 mM EDTA, 1% Triton-X 100, 0.1% sodium deoxycholate), LiCl Buffer (100 mM Tris-HCl, pH 8.0, 500 mM LiCl, 1% NP-40, 1% sodium deoxycholate), and 10 mM Tris-HCl, pH 8.0 at 4° C.

Tagmentation reaction (30 μl): 20 mM Tris-HCl, pH 7.5, 5 mM MgCl2, 10% dimethylformamide, and 1× Tagmentation Mix (final concentration: 1.25 μM Tn5, 5% glycerol, 750 nM adapter) was incubated for 30 minutes at 37° C. Following incubation, the resin was washed twice with Guanidine-hydrochloride Buffer (50 mM Tris-HCl, pH 7.5, 500 mM guanidine-hydrochloride, 2 mM EDTA, 1% Triton-X 100, 0.1% sodium deoxycholate) for 5 minutes at 37° C., then once with LiCl Buffer and 10 mM Tris-HCl, pH 8.0 at 4° C.

Fill-in reaction (30 μl): 10 U phi29 polymerase (NEB), 1× phi29 reaction buffer (NEB), 2× (200 μg/ml) BSA, and 165 μM dNTPs incubated for 20 minutes at 30° C.; then washed with 10 mM Tris-HCl, pH 8.0 at 4° C.

Kinase reaction (30 μl): 10 U T4 PNK (NEB), 1×T4 DNA Ligase Buffer (NEB), and 2×BSA incubated for 15 minutes at 37° C.; then washed with 10 mM Tris-HCl, pH 8.0 at 4° C.

λ exonuclease digestion (100 μl): 20 Uλ exonuclease (NEB), 1×λ exonuclease reaction buffer (NEB), 0.1% Triton-X 100, and 5% DMSO incubated for 30 minutes at 37° C.; then washed with 10 mM Tris-HCl, pH 8.0 at 4° C.

RecJ_{f} exonuclease digestion (100 μl): 75 U RecJ_{f} exonuclease (NEB), 2×NEBuffer 2, 0.1% Triton-X 100, and 5% DMSO incubated for 30 minutes at 37° C.; then washed with 10 mM Tris-HCl, pH 8.0 at 4° C.

Elution from resin and reverse cross-linking (40 μl): 30 μg Proteinase K, 25 mM Tris-HCl, pH 7.5, 2 mM EDTA, 200 mM NaCl, and 0.5% SDS incubated for 16 hours at 65° C.

The supernatant was then transferred to a new tube and purified with Agencourt AMPure magnetic beads (Beckman Coulter) following manufacturer's instructions.

The sample was eluted from the AMPure beads in 10 μl of water, and the following enzymatic steps were carried out in solution.

Primer extension (total reaction volume 20 μl): To the resuspended sample was added 1×phi29 reaction buffer, 2×BSA, 100 μM dNTPs, and 0.5 μM ME sequence oligonucleotide (total 9 μl) and incubated for 5 minutes at 95° C., then 10 minutes at 45° C. to allow the oligo time to anneal. The sample was shifted to 30° C. before adding 10 U phi29 polymerase (1 μl) and incubating for 20 minutes at 30° C.; then for 10 minutes at 65° C. to inactivate, and shifted to 37° C.

A-tailing reaction (total reaction volume 30 μl): To the primer extension reaction was added 10 U Klenow Fragment, −exo (NEB), 1×NEBuffer 2, 100 μM dATP (total 10 μl) and incubated for 30 minutes at 37° C., then for 20 minutes at 75° C. to inactivate, and shifted to 25° C.

Second adapter ligation (total reaction volume 40 μl): To the A-tailing reaction was added 2,000 U T4 DNA ligase (enzymatics), 1×NEBNext Quick Ligation Buffer (NEB), 375 nM adapter (ExA1-58/13) and incubated for 1 hour at 25° C.

The ligation reaction was then purified with AMPure beads and resuspended in 15 μl of water.

PCR amplification (total reaction volume 40 μl): To the resuspended DNA was added 2 U Phusion Hot Start polymerase (Thermo scientific), 1× Phusion HF Buffer (Thermo scientific), 200 μM dNTPs, 500 nM each primer (P1.3 and NexA2-iNN) and amplified for 18 cycles (20 second at 98° C. denature, 1 minutes at 52° C. annealing, 1 minutes at 72° C. extension). A quarter of the reaction was amplified for an additional six cycles (24 total) and the presence of libraries was determined by electrophoresis on a 2% agarose gel.

Size selection: 200 to 500 bp PCR products were gel-purified from a 2% agarose gel using the QIAquick Gel Extraction Kit (Qiagen).

ChIP-exo 4.0 and 4.1 (single-strand DNA ligation versions)

After immunoprecipitation, the following steps were carried out on the resin:

ChIP wash: the resin was washed sequentially with FA Lysis Buffer, NaCl Buffer, LiCl Buffer, and 10 mM Tris-HCl, pH 8.0 at 4° C.

End repair (50 μl): 7.5 U T4 DNA polymerase (NEB), 2.5 U DNA Polymerase I (NEB), 25 U T4 PNK, 1×T4 DNA Ligase Buffer, and 390 μM dNTPs incubated for 30 minutes at 12° C.; then washed with 10 mM Tris-HCl, pH 8.0 at 4° C.

λ exonuclease digestion (100 μl): 20 Uλ exonuclease, 1×λ exonuclease reaction buffer, 0.1% Triton-X 100, and 5% DMSO incubated for 30 minutes at 37° C.; then washed with 10 mM Tris-HCl, pH 8.0 at 4° C.

RecJf exonuclease digestion (100 μl): 75 U RecJ_{f} exonuclease, 2×NEBuffer 2, 0.1% Triton-X 100, and 5% DMSO incubated for 30 minutes at 37° C.; then washed with 10 mM Tris-HCl, pH 8.0 at 4° C.

First adapter ligation: First adapter ligation was performed using either ssDNA ligation or splint ligation.

ssDNA ligation (40 μl): 1,200 U T4 DNA ligase, 1×T4 DNA Ligase Buffer, and 375 nM single-strand adapter (ExA1-58-N5) incubated for 1 hour at 25° C.; then washed with 10 mM Tris-HCl, pH 8.0 at 4° C.

splint ligation (40 μl): 1,200 U T4 DNA ligase, 1×T4 DNA Ligase Buffer, and 375 nM adapter (ExA2.1-N5/ExA2.1-20) incubated for 1 hour at 25° C.; then washed with 10 mM Tris-HCl, pH 8.0 at 4° C.

Elution from resin and reverse cross-linking (40 μl): 30 μg Proteinase K, 25 mM Tris-HCl, pH 7.5, 2 mM EDTA, 200 mM NaCl, and 0.5% SDS incubated for 16 hours at 65° C.

The supernatant was then transferred to a new tube and purified with Agencourt AMPure magnetic beads (Beckman Coulter) following manufacturer's instructions.

The sample was eluted from the AMPure beads in 20 μl of water, and the following enzymatic steps were carried out in solution.

Second adapter ligation: Second adapter ligation was performed using either ssDNA ligation or splint ligation.

ssDNA ligation (total reaction volume 40 μl): To the resuspended DNA was added 1,200 U T4 DNA ligase, 1×T4 DNA Ligase Buffer, 375 nM adapter (ExA2.1-N5/ExA2.1-20) and incubated for 1 hour at 25° C.

splint ligation (total reaction volume 40 μl): To the resuspended DNA was added 1,200 U T4 DNA ligase, 1×T4 DNA Ligase Buffer, 375 nM adapter (ExA1-58/ExA1-SSL_N5) and incubated for 1 hour at 25° C.

The ligation reaction was then purified with AMPure beads and resuspended in 15 μl of water.

PCR amplification (total reaction volume 40 μl): To the resuspended DNA was added 2 U Phusion Hot Start polymerase (Thermo scientific), 1× Phusion HF Buffer (Thermo scientific), 200 μM dNTPs, 500 nM each primer (P1.3 and NexA2-iNN) and amplified for 18 cycles (20 second at 98° C. denature, 1 minutes at 52° C. annealing, 1 minutes at 72° C. extension). A quarter of the reaction was amplified for an additional six cycles (24 total) and the presence of libraries was determined by electrophoresis on a 2% agarose gel.

Size selection: 200 to 500 bp PCR products were gel-purified from a 2% agarose gel using the QIAquick Gel Extraction Kit (Qiagen).

As a note, ChIP-exo 4.0/4.1 incorporated a universal Read_2 adapter, with the barcode added later during PCR with long primers. Whenever long PCR primers were used in a library construction that involved lambda exonuclease digestion, the libraries suffered from low yield and high adapter dimers. Therefore the experiments described used full-length adapters and minimum length PCR primers.

ChIP-Exo 5.0

After immunoprecipitation, the following steps were carried out on the resin:

ChIP wash: the resin was washed sequentially with FA Lysis Buffer, NaCl Buffer, LiCl Buffer, and 10 mM Tris-HCl, pH 8.0 at 4° C.

A-tailing reaction (50 μl): 15 U Klenow Fragment, −exo (NEB), 1×NEBuffer 2, and 100 μM dATP incubated for 30 minutes at 37° C.; then washed with 10 mM Tris-HCl, pH 8.0 at 4° C.

First adapter ligation and kinase reaction (45 μl): 1,200 U T4 DNA ligase, 10 U T4 PNK, 1×NEBNext Quick Ligation Buffer, and 375 nM adapter (ExA2 iNN/ExA2B) incubated for 1 hour at 25° C.; then washed with 10 mM Tris-HCl, pH 8.0 at 4° C.

Fill-in reaction (40 μl): 10 U phi29 polymerase, 1×phi29 reaction buffer, 2×BSA, and 180 μM dNTPs incubated for 20 minutes at 30° C.; then washed with 10 mM Tris-HCl, pH 8.0 at 4° C.

λ exonuclease digestion (50 μl): 10 Uλ exonuclease, 1×λ exonuclease reaction buffer, 0.1% Triton-X 100, and 5% DMSO incubated for 30 minutes at 37° C.; then washed with 10 mM Tris-HCl, pH 8.0 at 4° C.

Elution from resin and reverse cross-linking (40 μl): 30 μg Proteinase K, 25 mM Tris-HCl, pH 7.5, 2 mM EDTA, 200 mM NaCl, and 0.5% SDS incubated for 16 hours at 65° C.

The supernatant was then transferred to a new tube and purified with Agencourt AMPure magnetic beads (Beckman Coulter) following manufacturer's instructions.

The sample was eluted from the AMPure beads in 20 μl of water, and the following enzymatic steps were carried out in solution.

Second adapter ligation (total reaction volume 40 μl): To the resuspended DNA was added 1,200 U T4 DNA ligase, 1×T4 DNA Ligase Buffer, 375 nM adapter (ExA1-58/ExA1-SSL_N5) and incubated for 1 hour at 25° C.

The ligation reaction was then purified with AMPure beads and resuspended in 15 μl of water.

PCR amplification (total reaction volume 40 μl): To the resuspended DNA was added 2 U Phusion Hot Start polymerase (Thermo scientific), 1× Phusion HF Buffer (Thermo scientific), 200 μM dNTPs, 500 nM each primer (P1.3 and P2.1) and amplified for 18 cycles (20 second at 98° C. denature, 1 minutes at 52° C. annealing, 1 minutes at 72° C. extension). A quarter of the reaction was amplified for an additional six cycles (24 total) and the presence of libraries was determined by electrophoresis on a 2% agarose gel.

Size selection: 200 to 500 bp PCR products were gel-purified from a 2% agarose gel using the QIAquick Gel Extraction Kit (Qiagen).

ChIP-seq 1-step

After immunoprecipitation, the following steps were carried out on the resin:

ChIP wash: the resin was washed sequentially with FA Lysis Buffer, NaCl Buffer, LiCl Buffer, and 10 mM Tris-HCl, pH 8.0 at 4° C.

Dual adapter ligation: ssDNA ligation (40 μl): 1,200 U T4 DNA ligase, 1×T4 DNA Ligase Buffer, and 375 nM of both adapters (ExA1-58/ExA1-SSL_N5 and ExA2.1-N5/ExA2.1-20) incubated for 1 hour at 25° C.; then washed with 10 mM Tris-HCl, pH 8.0 at 4° C.

Elution from resin and reverse cross-linking (40 μl): 30 μg Proteinase K, 25 mM Tris-HCl, pH 7.5, 2 mM EDTA, 200 mM NaCl, and 0.5% SDS incubated for 16 hours at 65° C.

The supernatant was then transferred to a new tube and purified with Agencourt AMPure magnetic beads (Beckman Coulter) following manufacturer's instructions.

The sample was eluted from the AMPure beads in 20 μl of water, and the following enzymatic steps were carried out in solution.

PCR amplification (total reaction volume 40 μl): To the resuspended DNA was added 2 U Phusion polymerase (Thermo scientific; note: the standard Phusion polymerase was used here instead of the Hot Start version), 1× Phusion HF Buffer (Thermo scientific), 200 μM dNTPs, 500 nM each primer (P1.3 and NexA2-iNN). Samples were then incubated at 72° C. for 5 minutes to fill-in the library, then 2 minutes at 95° C. to denature, followed by 18 amplification cycles (20 second at 98° C. denature, 1 minutes at 52° C. annealing, 1 minutes at 72° C. extension). A quarter of the reaction was amplified for an additional six cycles (24 total) and the presence of libraries was determined by electrophoresis on a 2% agarose gel.

Size selection: 200 to 500 bp PCR products were gel-purified from a 2% agarose gel using the QIAquick Gel Extraction Kit (Qiagen).

DNA Sequencing

High-throughput DNA sequencing was performed with a NextSeq 500 in paired-end mode producing 2×40 bp reads. Sequence reads were subsequently aligned to the yeast (sacCer3) and human (hg19) genomes using bwa-mem (v0.7.9a)23. Aligned reads were filtered to remove non-unique alignments and PCR duplicates. PCR duplicates were defined as sequence reads possessing identical Read_1 and Read_2 sequences.

The Results of the Experiments are now described.

ChIP-Nexus (ChIP-Exo 2.0) Assessment

ChIP-nexus was published as an updated version of the original ChIP-exo protocol that reported increased efficiency of adapter ligation through use of CircLigase (He et al., (2015) Nature biotechnology, 33:395-401). CircLigase catalyzes the self-circularization of single-stranded (ss) DNA and was used to reduce the number of intermolecular adapter ligation steps from two to one (Table 2). This reduction is achieved by putting both Illumina adapter sequences on a single oligonucleotide separated by a BamHI restriction site. Following circularization, the BamHI digestion creates linearized libraries that are suitable for DNA sequencing. Initially, the overall utility of ChIP-nexus (ChIP-exo 2.0) was evaluated as a replacement for ChIP-exo 1.1.

TABLE 2
Comparison of steps used in assay variants.

| Descriptive name | ChIP-exo | | ChIP-nexus | ChIP-tag-exo | | |
|---|---|---|---|---|---|---|
| Unique aspect | Original | | Circular | Tagmentation | | |
| | | | ligation | | | |
| Hands-on time | 12 hours | | 10.5 hours | 9 hours | | |
| Version | 1.0 | 1.1 | 2.0 | 3.0 | 3.1 | 3.2 |

| ChIP | ChIP | | ChIP | ChIP |
|---|---|---|---|---|
| First Adapter | Polish | | Polish | Tagmentation |
| Ligation | Kinase | | Kinase | |
| | A-tailing | | A-tailing | |
| | Adapter | Adapter | Adapter | |
| | ligation | ligation | ligation | |
| | (SOLiD) | (Illumina) | | |

| Exonuclease | Fill-in | Fill-in | Fill-in | | |
|---|---|---|---|---|---|
| Treatment | Polish | Polish | Polish | | |
| | Kinase | | Kinase | | |
| | Lambda exonuclease | Lambda exonuclease | Lambda exonuclease | | |
| | RecJ exonuclease | RecJ exonuclease | RecJ exonuclease | | |
| | Reverse XL/ProK | Reverse XL/ProK | Reverse XL/ProK | | |
| | AMPure | AMPure | AMPure | | |
| Second Adapter | Primer extension | Circularization | Primer | Adapter | Adapter |
| Ligation | | | extension | Ligation | ligation |
| | A-tailing | | A-tailing | (splint) | (ssDNA) |
| | Adapter ligation | BamH1 | Adapter | | |
| | | digestion | ligation | | |

| | | | |
|---|---|---|---|
| | AMPure | Ethanol | AMPure |
| | | precipitation | |
| PCR | PCR | PCR | PCR |

TABLE 3
Comparison of steps used in assay variants.

| Descriptive name | ChIP-SSL-exo | | ChIP-exo 5.0 | ChIP-seq 1-step |
|---|---|---|---|---|
| Unique aspect | ssDNA, | | Splint adapter | Splint adapter |
| | splint adapter | | | |
| Hands-on time | 6 hours | | 6.5 hours | 3.5 hours |
| Version | 4.0 | 4.1 | 5.0 | 1.0 |
| ChIP | ChIP | | ChIP | ChIP |
| First Adapter | Polish | | A-tailing | Adapter ligation |
| Ligation | Lambda exonuclease | | Adapter ligation/Kinase | (splint) |
| Exonuclease | Adapter | Adapter | Fill-in | |
| Treatment | ligation | ligation | Lambda exonuclease | |
| | (ssDNA) | (splint) | | |

| | | | |
|---|---|---|---|
| | Reverse XL/ProK | Reverse XL/ProK | Reverse XL/ProK |
| | AMPure | AMPure | AMPure |
| Second Adapter | Splint ligation | Splint ligation | PCR |
| Ligation | AMPure | AMPure | |
| PCR | PCR | PCR | |

In a completed ChIP-nexus library, a five bp random barcode and a four bp static barcode are incorporated immediately 3′ to where sequencing begins and immediately 5′ to the lambda exonuclease stop point (FIG. 1A). These are the first nine nucleotides of the first sequencing read (representing a ChIP-nexus “tag”), which are used to computationally remove PCR-duplicates and assess library quality. In evaluating the published ChIP-nexus data, it was noted that a significant number of sequencing tags (ranging from 20 to 95% for individual samples) were discarded because of poor barcode quality (He et al., (2015) Nature biotechnology, 33:395-401). This result was experimentally confirmed using the ChIP-nexus protocol. This represents a substantial loss of data.

Next, the basis for this data loss was evaluated. By definition, every sequencing read that passed the quality control filters contained the nucleotides CTGA in positions 6 to 9 of Read_1 (FIG. 1B, left panel). In reads that failed to pass filter, a sequential loss of nucleotides was observed in the static barcode (A>G>T>C decrease in peak amplitude, FIG. 1B, right panel). The start of this progression is internal to the completed library (i.e., not where sequencing begins), which is difficult to reconcile. However, these sequences reside at the end of the adaptor prior to circularization. Without being bound by theory, two possible sources were suggested for this loss: 1) incomplete oligo synthesis (these sequences were synthesized last, making them the least efficiently incorporated), or 2) T4 DNA polymerase end-trimming that occurs immediately before lambda exonuclease treatment. End-trimming is intended to create blunt-end DNA via the strong 3′ to 5′ exonuclease activity of T4 DNA polymerase (FIG. 1C). Overdigestion would result in preferential loss of A>G>T>C as seen in FIG. 1B, and illustrated in step 4b of FIG. 1C. This analysis indicates that the ChIP-nexus assay results in a substantial loss of data. While deeper sequencing could compensate, this incurs a higher financial cost. Moreover, the amount of CircLigase used in comparison to traditional T4 DNA ligase results in a ˜10-fold increase in the per-sample cost. ChIP-nexus also requires the additional enzymatic step of BamH1 digestion. Without being bound by theory, it was concluded that ChIP-nexus does not substantially improve the costs or technical difficulty of the ChIP-exo assay. In an effort to improve ChIP-exo, each step of library construction was revisited.

Tn5 Tagmentation-Based ChIP-Exo 3.0

To simplify the addition of adapters during library construction, DNA ligase was replaced with a hyperactive mutant Tn5 transposase (Goryshin et al., (1998) J Biol Chem, 273:7367-7374). This tagmentation reaction has been used to construct libraries for shotgun genome sequencing, chromatin accessibility (ATAC-seq), and ChIP-seq of transcription factors (ChIPmentation) (Adey et al., (2010) Genome Biol 11:R119; Schmidl et al., (2015) Nat Methods, 12:963-965; Buenrostro et al., (2015) Curr Protoc Mol Biol, 109:21 29 21-29). In ChIPmentation, chromatin is first fragmented by sonication, then immunoprecipitated and tagmented while on the resin. Tn5 dimers bind a pair of 19 bp DNA recognition sequences. An optimized version of the recognition sequence has been incorporated into the Illumina Nextera sequencing adapters. The mutant Tn5 inserts one end of each 19 bp DNA recognition sequence into genomic DNA at reduced target sequence specificity. This fragments the chromatin.

In an effort to develop a cost-effective tagmentation-based ChIP-exo assay (version 3.0), a Tn5 E. coli expression vector was constructed housing the E54K, E110K, P242A, and L372P mutations. These mutations create a hyperactive Tn5 that binds normally to its 19 bp recognition sequence, but has less sequence specificity for insertional targeting (Reznikoff, (2003) Mol Microbiol, 47:1199-1206). An N-terminal His6-tag was included for purification purposes. The three-step purification produced a high-active enzyme that was >95% pure (FIG. 2).

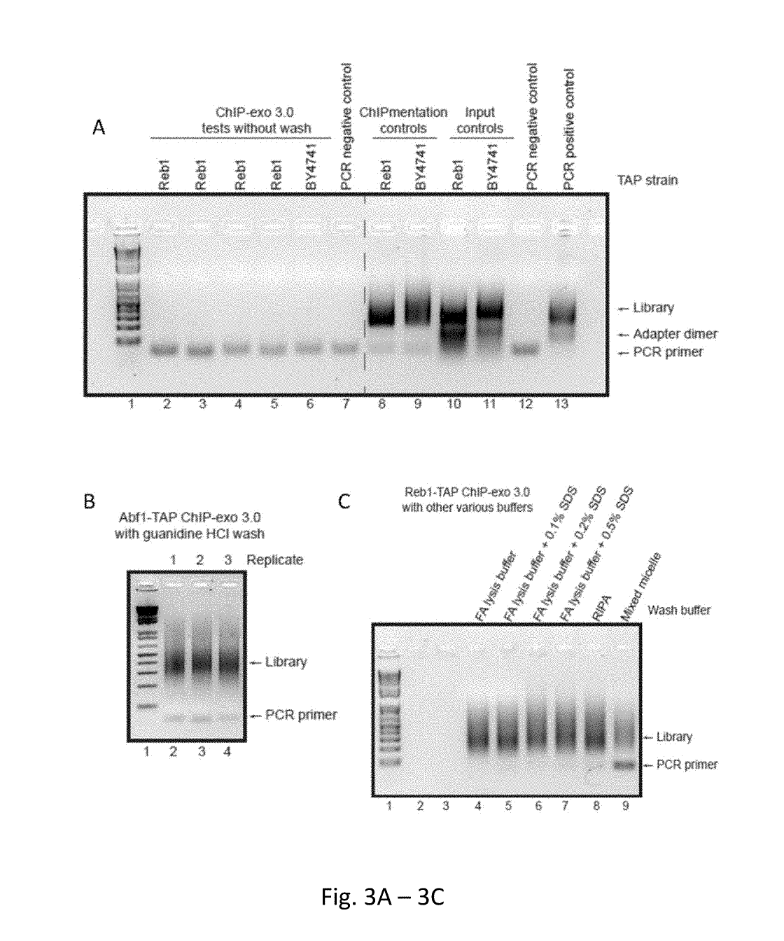

In ChIP-exo 3.0, immunoprecipitated chromatin was tagmented while on beads, as in ChIPmentation (Schmidl et al., (2015) Nat Methods, 12:963-965). At this point, it was necessary to remove the spent Tn5 that remained bound to the product DNA (Goryshin et al., (1998) J Biol Chem, 273:7367-7374), while maintaining the protein-DNA cross-links and protein-antibody interaction. Using standard wash buffers, without an additional treatment to remove spent Tn5, no library was detected (FIG. 3A, lanes 2-7). This is distinct from tagmentation, as the input controls did produce libraries (FIG. 3A, lanes 8-9). The reacted Tn5 could be stripped away using 500 mM guanidine hydrochloride buffer (see methods) (FIG. 3B). This allowed for efficient lambda exonuclease digestion equivalent to standard (version 1.1) ChIP-exo library construction (Rhee and Pugh, (2011) Cell, 147:1408-1419). Other chaotropic wash buffers were also successful (FIG. 3C). ChIP-exo 3.0 shortened the time for sample processing (Table 2), while maintaining library complexity (independent sampling events) seen in versions 1.1 and 2.0.

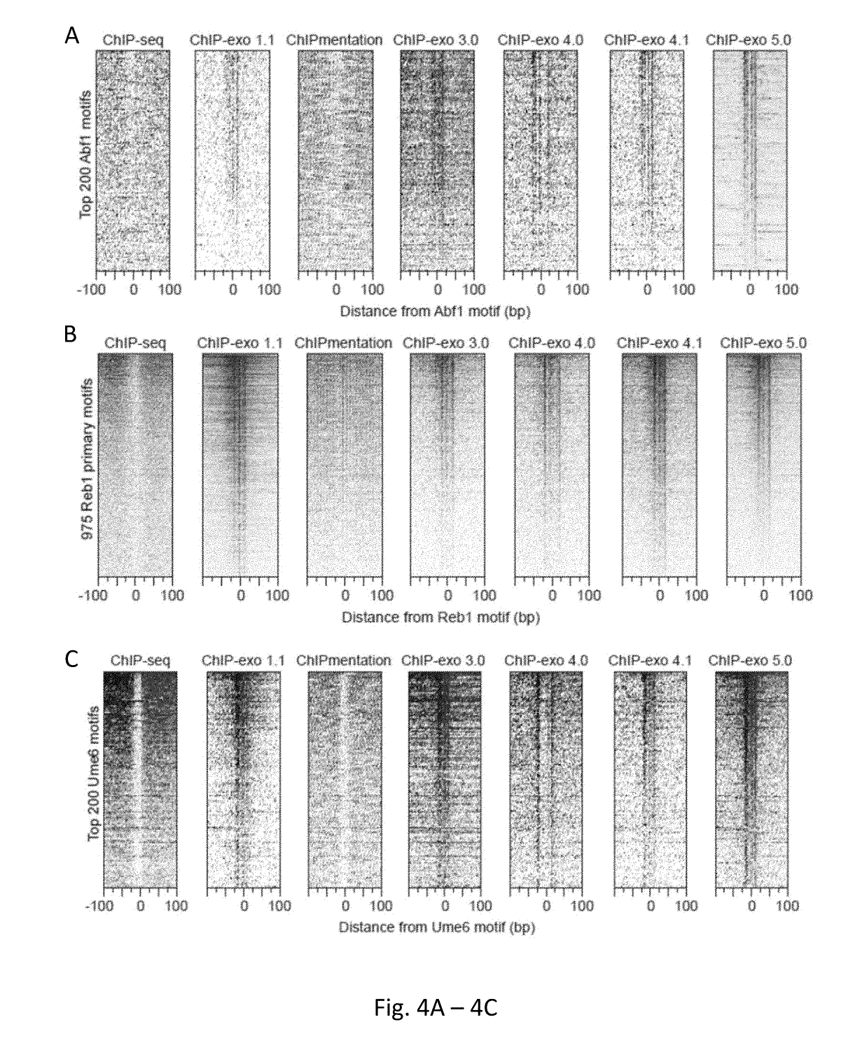

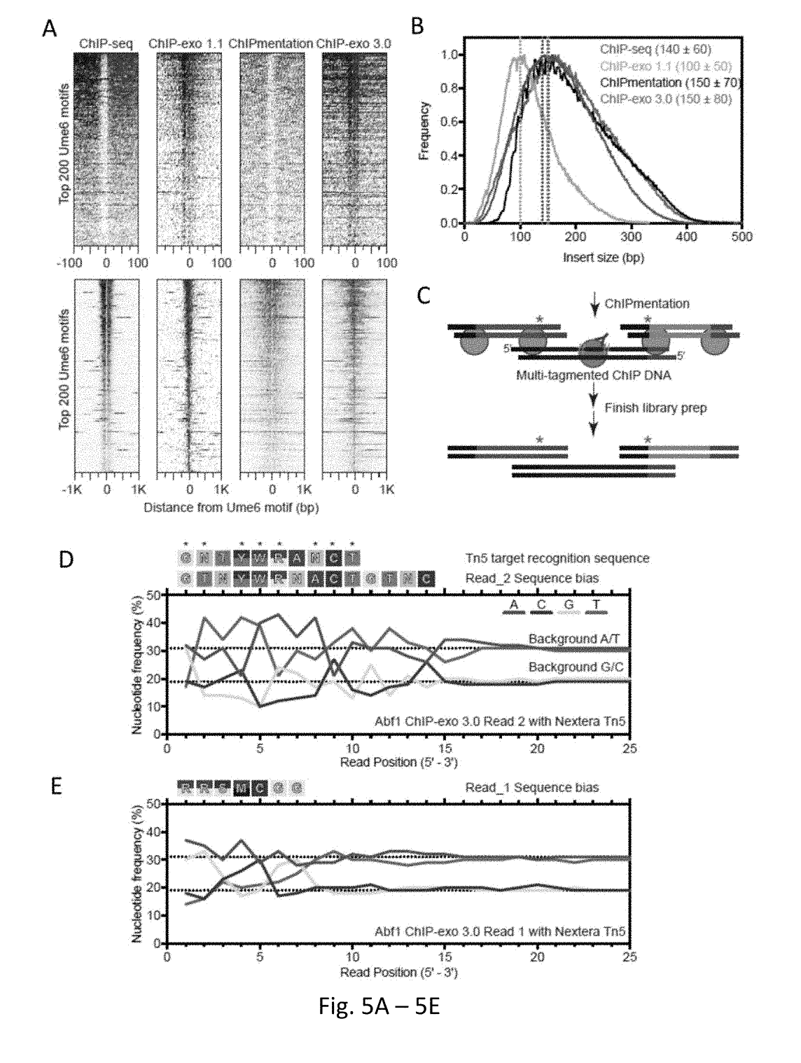

ChIP-exo 3.0 was tested on a set of yeast sequence-specific DNA binding proteins (Abf1, Reb1, and Ume6), all of which produced high quality data with ChIP-exo 1.0 (and 1.1). The same qualitative results were obtained with all tested factors (FIG. 4); but for simplicity the focus is on Ume6. Ume6 is a sequence-specific transcription factor that represses transcription of early meiotic genes through recruitment of chromatin remodelers (Yadon et al., (2013) Mol Cell, 50:93-103). Genome-wide binding of Ume6 was assayed by ChIP-seq, ChIP-exo 1.1, ChIPmentation, and ChIP-exo 3.0 (and also subsequent versions). The resulting tag 5′ ends were plotted around the top 200 bound Ume6 motifs (FIG. 5A, the left hand side of each graph represents those on the reference motif strand, and the right hand side of each graph represents those on the opposite strand). ChIP-exo 3.0 resulted in the same exonuclease stop sites at Ume6 motifs (vertical “stripes”) as seen with version 1.1 (Supplementary FIG. 3a). Thus, high resolution ChIP-exo can be conducted on tagmented chromatin.

Analysis of ChIP-Exo 3.0 and ChIPmentation

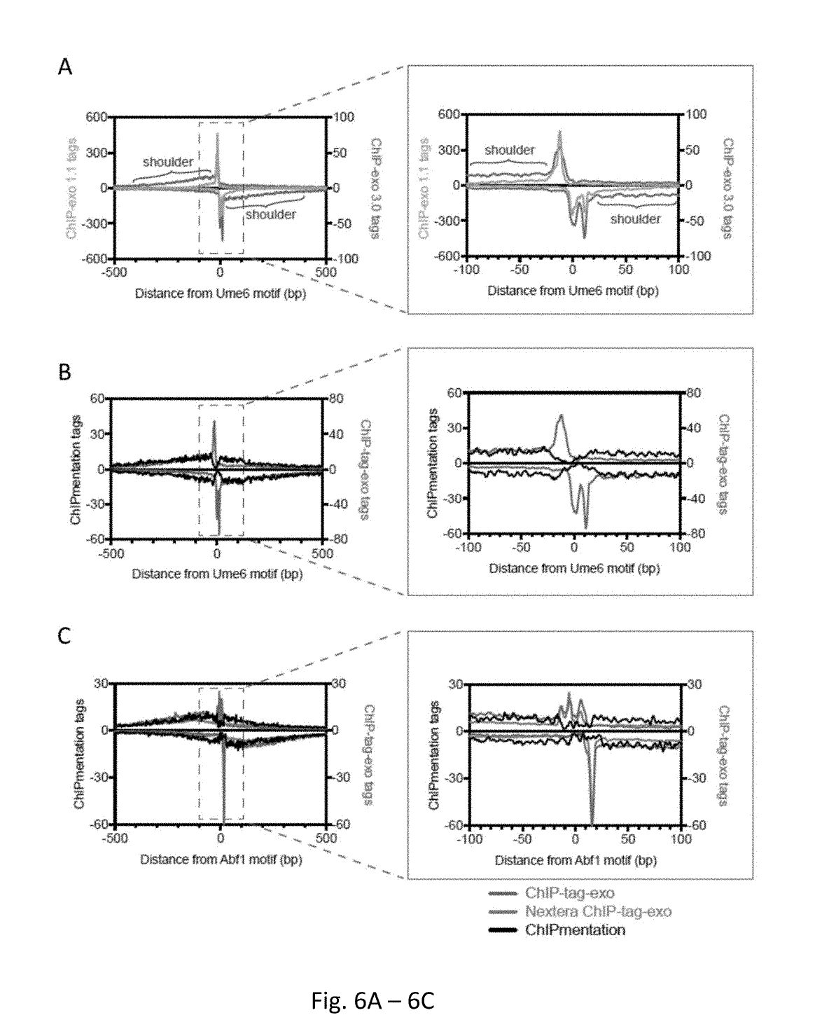

In ChIP-exo 3.0, a substantial proportion of tag 5′ ends mapped hundreds of bp beyond the core exonuclease stop sites, which was largely absent in version 1.1 (broad shouldering in FIG. 6A). Without being bound by theory, it was surmised that this may reflect ineffective exonuclease digestion of a portion of the tagmented DNA molecules, whereas other molecules were digested effectively. Additional stringent washes of the chromatin did not improve the outcome. If the stringent washes failed to completely remove Tn5, then the residual Tn5 may be blocking exonuclease digestion. This makes ChIP-exo 3.0 relatively less efficient from a sequencing yield perspective, in that most specific tags were lower resolution than version 1.1.

Surprisingly, the broad shouldering in version 3.0 was at least as broad as in ChIP-seq and equivalent to ChIPmentation (flanks in FIG. 5A, lower set of panels; also FIG. 5B). Increased concentrations of Tn5/adaptor complexes, within limits of practicality, did not appreciably reduce the broad shouldering. Without being bound by theory, it was expected that the library size of ChIPmentation would be marginally shorter than ChIP-seq due to Tn5 fragmentation of the already-sonicated chromatin. It was also expected that ChIP-exo 3.0 would be ˜50 bp shorter than ChIPmentation, due to shortening by the exonuclease. However, the main observed difference was an increase in abundance of larger fragments. Without being bound by theory, this was attributed to preferential tagmentation of longer DNA molecules due to more opportunities for Tn5 binding (Steiniger et al., (2006) Nucleic Acids Res, 34:2820-2832). Many short fragments may have gone unreacted. Consequently, tagmentation created a bias towards larger library fragment size, which may create a bias in binding site detection.

A second surprising finding was that, unlike version 1.1 and ChIP-seq, previously reported Tn5-based (ChIPmentation) assays produced substantial amounts of tag 5′ ends that mapped downstream (more 3′) of the reference motif (FIG. 6A and FIG. 6B). Thus, the ChIPmentation heatmap did not produce a segregated left/right pattern of tag 5′ ends, as seen in the ChIP-seq and ChIP-exo 1.1 heatmaps (FIG. 5A), making a Tn5-based approach for high resolution mapping non-obvious. Without being bound by theory, it is hypothesized that this may be due to multiple Tn5 hits occurring on the same ChIP DNA molecule, but with the multiple DNA fragments being held together noncovalently via Tn5-DNA product complexes (FIG. 5C). This would allow tag 5′ ends to map downstream of the cross-link. In contrast, in normal ChIP-seq the 5′ ends of the double-stranded DNA reside on opposite sides of the ChIP peak (left panel in FIG. 5A). Thus, when analyzed by tag 5′ ends, ChIPmentation produced a resolution below that of ChIP-seq.

To test whether the tagmentation issues identified above were particular to the in-house Tn5 preparation or are also present in commercially-available Tn5, the Nextera DNA Library Preparation Kit was used. For these particular experiments, the two preparations were compared against the sequence-specific DNA binding protein Abf1. Significant shoulders were somewhat reduced, but still prominent with the commercial Tn5 (FIG. 6C). Although the hyperactive Tn5 was engineered to be less sequence-specific in insertional target site selection, both the in-house Tn5 preparation and the Nextera Tn5 retained a strong cleavage preference for the wildtype target recognition sequence (FIG. 5D). This targeting bias significantly skewed the nucleotide frequency at the 5′ end of tagmented sequencing reads. The lambda exonuclease treatment eliminated most of the bias in ChIP-exo 3.0 reads (FIG. 5E), and masked the intrinsic biased imposed by Tn5. Taken together, ChIP-exo 3.0 is technically simpler than 1.0/2.0, and provides equivalent location resolution.

ChIP-Exo 4.0 and 4.1 Development (Single-Stranded DNA Ligation Versions)

To further reduce the number of steps and increase efficiency for standard adapter ligation, strategies employing single-stranded (ss) DNA ligation were evaluated. In ChIP-exo 1.0/1.1, double-stranded ChIP DNA is blunt-ended, then A-tailed prior to ligation of the first adapter by T4 DNA ligase. This reaction is inefficient, likely due to the numerous (four) enzymatic steps. However, efficient intermoleculer ligation has been described between the 3′ ends of ssDNA and the 5′ ends of a single-stranded adapter oligo using either CircLigase (Gansauge and Meyer, (2013) Nat Protoc, 8:737-748) or T4 DNA ligase (Kwok et al., (2013) Anal Biochem, 435:181-186).

To efficiently incorporate ssDNA ligation into ChIP-exo, while retaining use of the more cost-effective T4 DNA ligase (compared to CircLigase), the order of the enzymatic steps was rearranged such that lambda exonuclease digestion occurred before DNA ligation (Table 2). In this way, the assay was able to take advantage of the ssDNA produced by lambda exonuclease. However, there was uncertainty as to whether the exonuclease would work efficiently on sonicated unpolished ends, as they might contain 3′ phosphates that are not efficient substrates for the exonuclease. The polarity of the resected 5′ ends generated by lambda exonuclease was exploited to direct end-specific hybridization-based DNA ligation (FIG. 7A). In this case, the Read_1 adapter was a ssDNA oligo that contained a random pentamer at its 3′ end. Hybridization of the random pentamer to the ssDNA that was complementary to the resected strand, and adjacent to the exonuclease stop site 5′ end produced efficient ligation. Other oligomers having random 4- and 6-mers were tested and found to be successful in construction of input libraries (Table 4). This ligation scenario differs from other reported single-stranded ligation reactions (Kwok et al., (2013) Anal Biochem, 435:181-186 and version 4.1) in the following ways: a) the DNA is immobilized (and thus subject to diffusion and reactivity limits), and b) utilization of a single-stranded adapter, as opposed to adapters that form secondary structures (hairpins, double-stranded oligos) in which one end is ligated and the other end provides specificity and affinity through complimentary base-pairing.

TABLE 4
Sequencing statistics for input library produced using oligos
with varying lengths of random single stranded overhangs.

| Sample description | | Unique | | Aligned | | Median |
|---|---|---|---|---|---|---|
| (input library | | Aligned | | Deduplicated | % Aligned | Insert Size |
| with . . .) | Total Tags | Tags | % Aligned | Tags | Deduplicated | (bp) |
| random 4-mer | 4,090,674 | 2,301,023 | 56% | 2,202,148 | 54% | 136 |
| random 5-mer | 3,899,269 | 1,690,507 | 43% | 1,633,577 | 42% | 133 |
| random 6-mer | 3,895,489 | 1,538,543 | 39% | 1,463,566 | 38% | 134 |
| random 4, 5, and | 3,641,784 | 1,578,694 | 43% | 1,531,811 | 42% | 136 |
| 6-mers | | | | | | |

In the adopted scheme, the ligation reaction involves juxtaposing the 3′ end of the adapter oligo next to the resected 5′ end of genomic DNA, wherein the sequence complementary to the resected (hydrolyzed) DNA provides specificity and affinity for the single-strand oligo adapter. The incorporated random pentamer sequence represents the first five positions of the sequencing read, making the exonuclease stop site appear 5 bp more 5′ than in other versions of ChIP-exo. This is version 4.0. The ligation scheme differs from other ssDNA ligation descriptions in being conducted on immobilized DNA rather than in solution, and involving adapter ligation to the 5′ end of genomic DNA rather than its 3′ end.

The immobilized nature of low concentrations of genomic DNA and its lack of diffusibility rendered it uncertain as to whether an oligo N-mer adapter would have sufficient specificity and affinity to promote efficient single-stranded ligation. Given that there is a trade-off between stabilization (longer N-mers being more stable) and specificity (longer N-mers being more selective, and thus less efficient), it was not obvious that the balance would reside in a cost-efficient range or that side-reactions such as adapter dimers would not dominate (and inhibit) the reaction.

In an alternative scenario (ChIP-exo 4.1), the first ligation to the 3′ ssDNA end of the resected ChIP-exo DNA was conducted while the DNA remained immobilized on resin (FIG. 7B). This was performed by using a double-stranded Read_2 adapter having a random 3′ ssDNA pentamer overhang. Upon hybridization of this “splint” to the terminal five nucleotides of ChIP-exo ssDNA 3′ end, T4 DNA ligation of the complementary adapter strand to the ChIP-exo ssDNA 3′ end proceeded efficiently.

Although similar tests have been performed on single-stranded DNA in solution, there have been no parallel tests for immobilized single-stranded DNA using this type of splint ligation. The immobilized nature of the DNA would have the same potential non-obvious constraints indicated for version 4.0.

In both ChIP-exo schemes (4.0/4.1), following reversal of the formaldehyde cross-linking, the second adapter is attached using another splint ligation of proper polarity (Read_2 adapter for 4.0, and Read_1 adapter for 4.1). By using random pentamers to guide adapter ligation instead of standard A-tailing, ChIP-exo 4.0/4.1 eliminates nine enzymatic steps and nearly six hours of hands-on time from standard ChIP-exo.

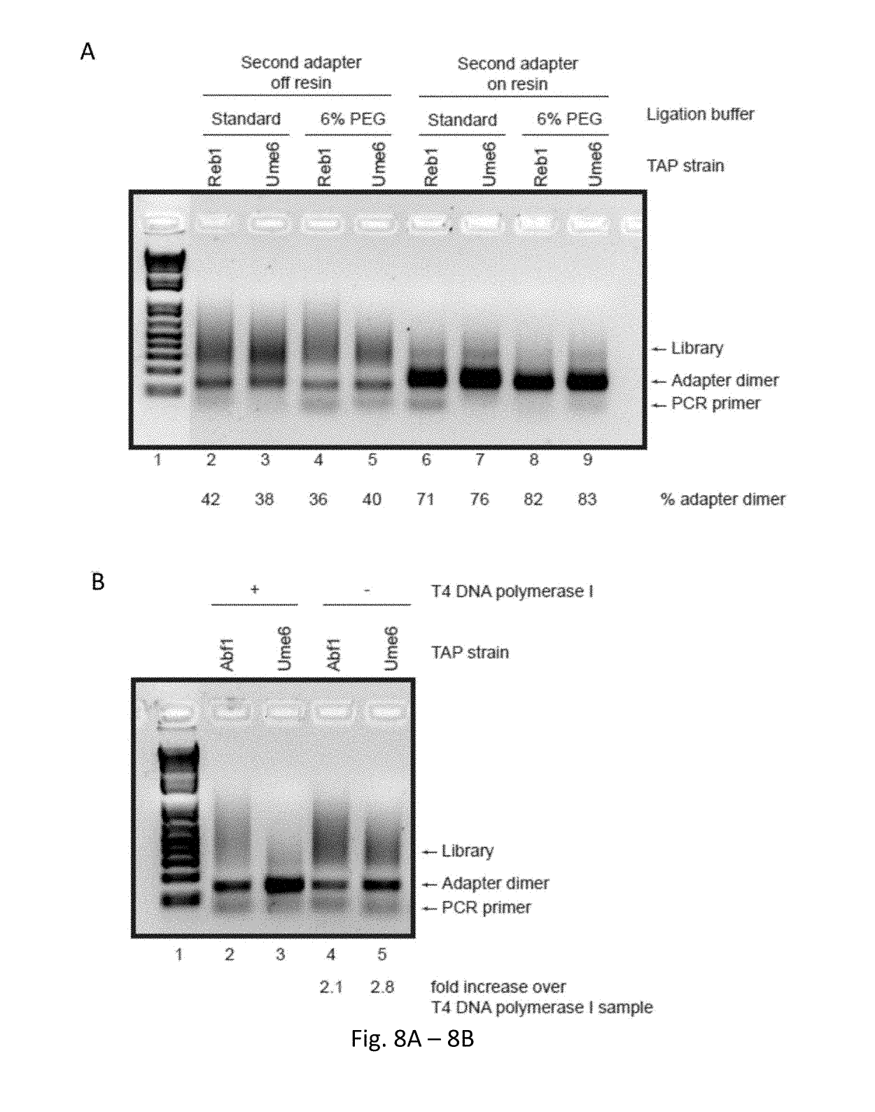

Second adapter ligation was initially attempted on immobilized DNA (prior to reversal of the formaldehyde crosslinking). However, this resulted in unacceptably high levels of adapter dimers (ligation of the first and second adapters) (FIG. 8A, lanes 6-9, compared to successful off-resin ligation, lanes 2-5). Adapter dimers take up sequencing bandwidth, and thus cause decreased efficiency as they incur the cost of sequencing without providing genomic sequence information. Therefore, second ligation was conducted after crosslinking reversal and DNA clean-up.

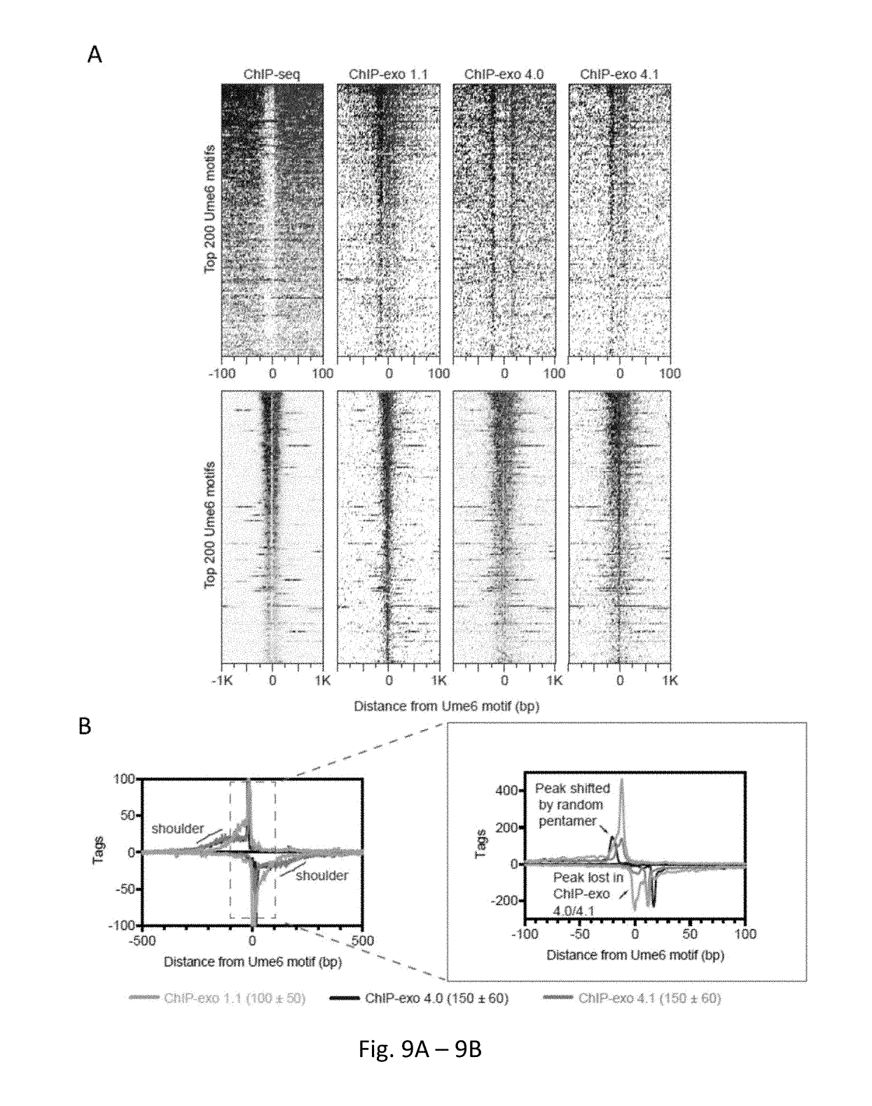

Much like ChIP-exo 3.0, it was found that 4.0/4.1 provided high resolution patterning of factor binding, but also contained significant amounts of low-resolution shouldering, presumably from incomplete exonuclease digestion (FIG. 9A and FIG. 4). Given the caveat that ChIP-exo peaks detected in version 4.0 are shifted five bp further away from the motif midpoint, the ChIP-exo 4.0/4.1 composite plots produced the same outer peaks (exonuclease stops) as ChIP-exo 1.1/3.0 (FIG. 9A, vertical stripes are farther apart in ChIP-exo 4.0 panel). However, a secondary inner peak on the motif-complementary strand in the Ume6 pattern was greatly reduced in version 4.0/4.1, but not 1.1/3.0 (FIG. 9B and FIG. 6A). Without being bound by theory, it was hypothesized that it might relate to an altered ligation efficiency that occurs at or near a particular motif sequence or cross-link. Nevertheless, ChIP-exo 4.0/4.1 represent two highly streamlined versions of ChIP-exo.

ChIP-Exo 5.0 Development

While ssDNA ligation in versions 4.0/4.1 significantly improved technical aspects of ChIP-exo, the relative level of undesirable “shouldering” (undigested ChIP DNA) was greater when compared to ChIP-exo 1.0/1.1. Without being bound by theory, it was hypothesized that the low shouldering in ChIP-exo 1.0/1.1 might be due to it having the first ligation step performed prior to exonuclease digestion, as this is the major distinction between versions 1 and 4. Conceivably, the early enzymatic steps may have created or selected for DNA molecules that are competent for exonuclease digestion. Therefore ChIP-exo 5.0 was developed which returned to the processing order specified in ChIP-exo 1.1, but tested the assumption that every step in ChIP-exo 1.0/1.1 is required, since those steps were based on theoretical expectations, rather than actual experimental testing.

The first enzymatic step in ChIP-exo is to create blunt ends from DNA fragmented by sonication, using T4 DNA polymerase (Table 2). Since T4 DNA polymerase possesses both 5′ to 3′ synthesis and 3′ to 5′ exonuclease activities, the reaction was carried out at 12° C. to balance these opposing activities. The widely-held assumption that T4 DNA polymerase would produce more ligatable blunt ends through synthesis than it eliminated through exonuclease activity was found to be incorrect, as removal of the T4 DNA polymerase polishing step increased the library yield (FIG. 8B). Consequently, in ChIP-exo 5.0 all polishing steps were removed. A-tailing by Klenow was maintained as it restored “shouldering” to acceptable levels, as seen in 1.0/1.1.

The next two steps after A-tailing involved T4 Kinase and T4 DNA ligase. Since both work well in the same buffer, they were combined into a single step, allowing both the ChIP DNA and adapter 5′ ends to be phosphorylated and ligated (despite the oligos being synthesized with a 5′ phosphate). Thus, the T4 Kinase improves efficiency but is not absolutely necessary. Should nonspecific dephosphorylation occur, the T4 Kinase would restore proper 5′ phosphates, which are required for ligation. The ligation buffer was altered to include polyethylene glycol, which as demonstrated elsewhere (He et al., (2015) Nat Biotechnol, 33:395-401), increased yield and decreased incubation times. RecJf exonuclease digestion was also removed. Its original purpose was to eliminate nonspecific ssDNA contaminants that might arise from lambda exonuclease digestion of contaminating double-stranded DNA. However, this step had no discernible impact on library quality. As a result of these improvements, five enzymatic steps and four hours of incubation time were eliminated from this part of the ChIP-exo 1.1 protocol. The remaining steps were performed as in ChIP-exo 4.1 (Table 2 and Table 3). The entirety of this streamlined procedure is ChIP-exo 5.0 (FIG. 10A).

With ChIP-exo 5.0, the same high quality libraries and data as 1.1 were obtained with only five enzymatic steps compared to the original thirteen. The reduction in steps also greatly increased library yield, as shown when the same ChIP reactions were split and libraries were constructed using either the 1.1 or 5.0 versions of the protocol. When equal amounts of library were sequenced, version 5.0 produced ten times more mapped tags than version 1.1, after removal of PCR duplicates (FIG. 10B). ChIP-exo 5.0 produced robust Reb1 data, even though the chromatin input in the immunoprecipitation was reduced five-fold relative to the published ChIP-exo 1.1 protocol. With the same amount of chromatin, ChIP-exo 1.1 barely registered Reb1 binding (FIG. 10C). The increased yield of ChIP-exo 5.0 also led to a higher rate of successful ChIPs for low-abundance factors such as Mcm1, Fkh1, Hap5, Hap2, and Nrg1 (FIG. 10D).

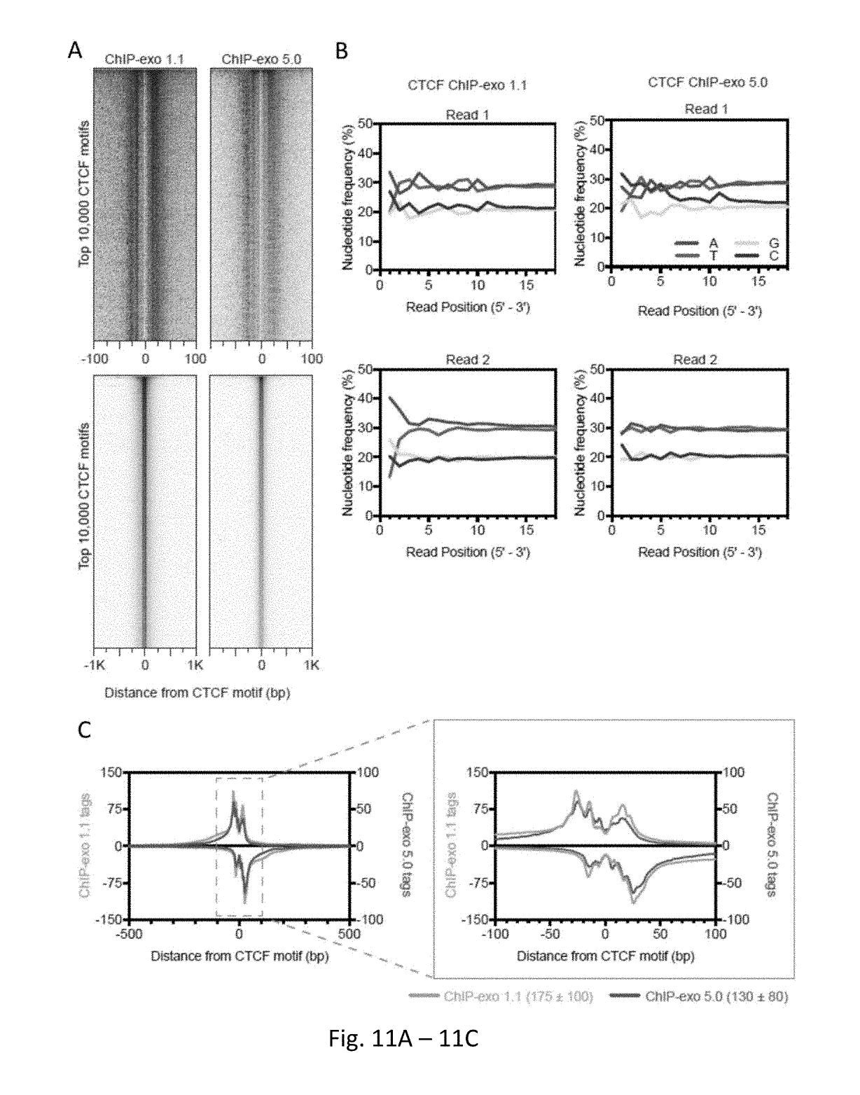

To demonstrate that the advantages of ChIP-exo 5.0 are not exclusive to yeast samples, ChIP-exo 5.0 was performed for CTCF in the mammalian K562 cells. Compared to ChIP-exo 1.1, ChIP-exo 5.0 produced equivalently high resolution CTCF exonuclease stop sites (FIG. 11A), with relatively little nucleotide bias near the ligated ends (FIG. 11B). Importantly, the shouldering that was evident in ChIP-exo 3.0/4.0/4.1 was greatly diminished (FIG. 11C). In every cell type tested, ChIP-exo 5.0 is a strict improvement over the ChIP-exo 1.1 method.

ChIP-Seq 1-Step

The simplicity of splint ligation demonstrated in ChIP-exo 4.0/4.1/5.0 prompted the consideration of its utility in ChIP-seq, where library construction might occur in one enzymatic step. In standard ChIP, chromatin is fragmented by sonication. This may result in a variety of “frayed” DNA ends with 5′ or 3′ ssDNA overhangs. In typical ChIP-seq library construction, these ends are blunted (made flush) and A-tailed through multiple enzymatic steps that include T4 DNA polymerase, E. coli DNA polymerase I, T4 polynucleotide kinase, and Klenow fragment (Johnson et al., (2007) Science, 316:1497-1502; Consortium, (2012) Nature, 489:57-74). Each step requires an inherent commitment of time and cost. As these steps have been utilized in ChIP-seq library construction for nearly ten years, it was not obvious that they could be replaced by a single-stranded ligation reaction, particularly since the immobilized DNA is not dissociated into single-stranded DNA. Moreover, co-incubation of Read_1 and Read_2 adapters, risked creating adapter dimer problems. Whether this would occur or not was unclear, as Read_1 and Read_2 adapters are quite different in end compatibility.

To test whether sonicated ChIP DNA could be directly ligated to adapters without blunt ending, resin-bound ChIP material was incubated simultaneously with two types of double-stranded adapters, either containing a random 3′ (sequenced as Read_1) or 5′ (sequenced as Read_2) pentamer overhang (FIG. 12A). Since the overhangs of Read_1 and Read_2 adapters have opposite polarity, the formation of unwanted Read_1-Read_2 adapter heterodimers is minimized. Unwanted homodimers (R1-R1 and R2-R2) form hairpins and thus do not amplify efficiently. Inclusion of other possible overhang adapter combinations, or conversion to forked adapters, produced an intractable amount of adapter dimers, and thus were not adopted. However, further optimization of reaction conditions might improve the utility of these alternative adaptors. Ligating one adapter, followed by a wash of the resin, then ligating the second adapter did not significantly improve library quality. The “ChIP-seq 1-step” procedure, which involved a single ligation reaction followed by PCR amplification produced high-quality ChIP-seq libraries in human (FIG. 12B) and yeast (FIG. 12C).

The possibility that use of random pentamers might create a sequence-biased library at the first five positions of each read was considered. However, contrary to this concern, the nucleotide bias for ChIP-seq 1-step was generally diminished compared to standard ChIP-seq (FIG. 12D). Thus ChIP-seq 1-step provides a simple alternative to the standard ChIP-seq with reduced cost and processing time and reduced ligation bias.

### Example 2

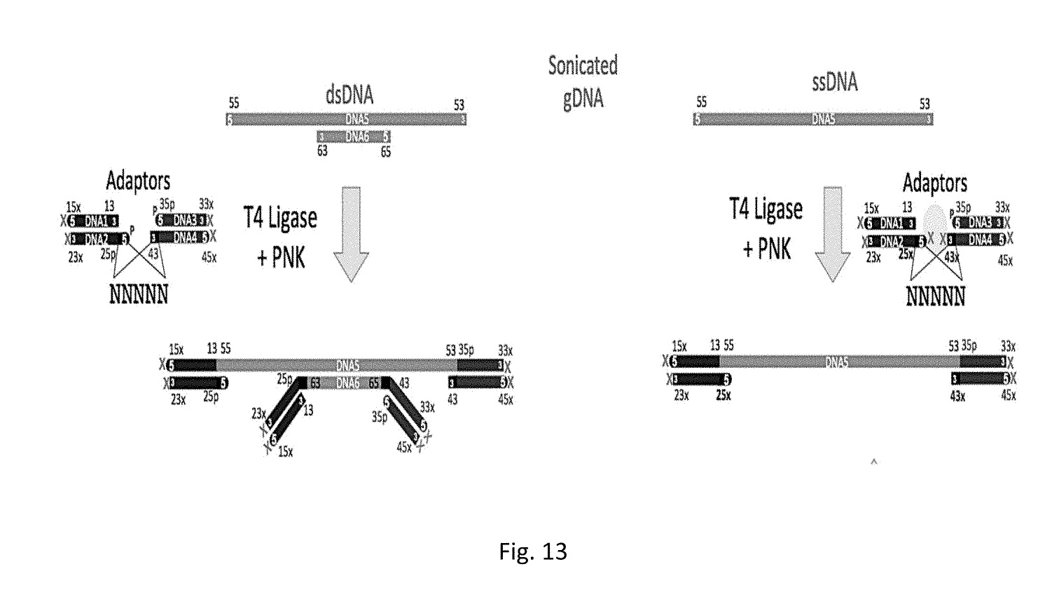

Various modified ChIP-seq and modified ChIP-exo methods of the invention are described with references to FIG. 13.

In one embodiment, the modified ChIP-seq and modified ChIP-exo methods of the invention provide methods of ligation between a DNA 3′-OH and a DNA 5′-Phosphate, wherein the DNA is immobilized.

In one embodiment, the DNA is used to detect where a polypeptide or protein is bound along the DNA sequence

In one embodiment, the protein or polypeptide is crosslinked to DNA as described in U.S. Pat. No. 8,367,334.

In one embodiment, DNA cleavage activity is applied to the immobilized crosslinked material.

In one embodiment, the 5′ and/or 3′ ends of the plurality of immobilized crosslinked DNA strands are separated or dissociated from the cleaved DNA strand(s) or nucleotide(s) AND NOT dissociated from its complementary strand(s) or nucleotide(s); i.e., 5′ and/or 3′ ends are double-stranded (FIG. 13, marks 65, 63). In one embodiment, the DNA cleavage activity includes sonication, reactive chemicals, radiation, and/or DNA cleaving enzymes such as an endonuclease or exonucleases (strand-specific or strand-nonspecific). Examples include, but are not limited to, Tn5 transposase, a strand-specific 5′-3′ exonuclease (lambda exonuclease), and permanganate/piperidine (PIP-seq). Exemplary methods include ChIP-exo 3.0, 5′ SS ligation (with or without constant-sequence complementary strand) (ChIP-exo 4.0), and a variation of ChIP-exo 4.0 wherein at least one adaptor molecule is ligated through a method of 3′ SS ligation.

In one embodiment, the 5′ and/or 3′ ends of the plurality of immobilized crosslinked DNA strands are separated from the cleaved DNA strand(s) or nucleotide(s) AND separated from its complementary strand(s) or nucleotide(s); i.e., is single-stranded (FIG. 13 marks 55, 53). In one embodiment, the DNA cleavage activity includes sonication, reactive chemicals, radiation, and/or DNA cleaving enzymes such as an endonuclease or exonucleases (strand-specific or strand-nonspecific), but additionally may include strand-separating activity achieved through physical (e.g. thermal), chemical (e.g. high pH), or enzymatic activity (e.g., helicase). Exemplary methods include methods in which at least one adaptor molecule is ligated through a method of 5′ or 3′ splint ligation, including but not limited to ChIP-exo 4.1, ChIP-exo 5.0 and One-step ChIP-seq.

In one embodiment, the DNA cleavage activity is not applied to the immobilized crosslinked material. In one embodiment, the DNA has free 5′ and/or 3′ ends that are NOT dissociated from its complementary strand(s) or nucleotide(s); i.e., 5′ and/or 3′ ends are double-stranded (FIG. 13 marks 63, 65). In one embodiment, the DNA cleavage activity includes sonication, reactive chemicals, radiation, and/or DNA cleaving enzymes such as an endonuclease or exonucleases (strand-specific or strand-nonspecific). Examples include, but are not limited to, Tn5 transposase, a strand-specific 5′-3′ exonuclease (lambda exonuclease), and permanganate/piperidine (PIP-seq). Exemplary methods include WhIP-exo, PB-exo, and PB-seq.

In one embodiment, the DNA has free 5′ and/or 3′ ends that are dissociated from its complementary strand(s) or nucleotide(s); i.e., 5′ and/or 3′ ends are single-stranded (FIG. 13 marks 55, 53). In one embodiment, the DNA cleavage activity includes sonication, reactive chemicals, radiation, and/or DNA cleaving enzymes such as an endonuclease or exonucleases (strand-specific or strand-nonspecific), but additionally may include strand-separating activity achieved through physical (e.g. thermal), chemical (e.g. high pH), or enzymatic activity (e.g., helicase). Examples include, but are not limited to, Tn5 transposase, a strand-specific 5′-3′ exonuclease (lambda exonuclease), and permanganate/piperidine (PIP-seq). Exemplary methods include WhIP-exo, PB-exo, and PB-seq.

In one embodiment, the protein or polypeptide is not crosslinked to DNA

In one embodiment, the DNA is not used to detect where a polypeptide or protein is bound along the DNA sequence.

In one embodiment, the DNA is not immobilized

In one embodiment, the adaptors have the following properties in a solution suitable for DNA ligation (FIG. 13):

DNA1: In one embodiment, the DNA1 adaptor molecule comprises a ligatable or unligatable 5′ end (15×). In one embodiment, the DNA1 adaptor molecule comprises a specified or unspecified DNA sequence. In one embodiment, the DNA1 adaptor molecule comprises a ligatable 3′ end (13). In one embodiment, the 3′ end (13) of the DNA1 adaptor molecule base-pairs (hybridizes) to the DNA2 adaptor molecule under ligation conditions

DNA2: In one embodiment, the DNA2 adaptor molecule comprises a ligatable (25p) or unligatable (25×) 5′ end. In one embodiment, the DNA2 adaptor molecule comprises a phoshorylated or unphosphorylated 5′ end (25p). In one embodiment, the DNA2 adaptor molecule comprises a plurality of nucleotides at the 5′-most N positions being essentially random (25p). In one embodiment, N>2. In one embodiment, N<10. In one embodiment, N=4, 5, or 6. In one embodiment, the DNA2 adaptor molecule comprises a specified or unspecified DNA sequence. In one embodiment, the DNA2 adaptor molecule comprises a ligatable or unligatable 3′ end (23×). In one embodiment, the DNA2 adaptor molecule base-pairs (hybridizes) to DNA1 under ligation conditions.

DNA3: In one embodiment, the DNA3 adaptor molecule comprises a ligatable 5′ end (35p). In one embodiment, the DNA3 adaptor molecule comprises a phoshorylated or unphosphorylated 5′ end (35p). In one embodiment, the DNA3 adaptor molecule comprises a specified or unspecified DNA sequence. In one embodiment, the DNA3 adaptor molecule comprises a ligatable or unligatable 3′ end (33×). In one embodiment, the 5′ end (35p) of the DNA3 adaptor base-pairs (hybridizes) to DNA4 under ligation conditions.

DNA4: In one embodiment, the DNA4 adaptor molecule comprises ligatable or unligatable 5′ end (45×). In one embodiment, the DNA4 adaptor molecule comprises a specified or unspecified DNA sequence. In one embodiment, the DNA4 adaptor molecule comprises a ligatable (43p) or unligatable (43×) 3′ end. In one embodiment, the DNA4 adaptor molecule comprises a plurality of nucleotides at the 3′-most N positions being essentially random (43p). In one embodiment, N>2. In one embodiment, N<10. In one embodiment, N=4, 5, or 6. In one embodiment, the DNA4 adaptor molecule base-pairs to DNA3 under ligation conditions.

In one embodiment, the target nucleic acid molecules have the following properties in a solution suitable for DNA ligation (FIG. 13):

DNA5: In one embodiment, the DNA5 sequence comprises a ligatable 5′ end (55) and a ligatable 3′ end (53). In one embodiment, the DNA5 sequence comprises a single-stranded 5′ end (55) and/or a single-stranded 3′ end (53). In one embodiment, the DNA5 sequence comprises a specified or unspecified DNA sequence.

DNA6: In one embodiment, the DNA6 sequence comprises a ligatable 5′ end (65) and a ligatable 3′ end (63). In one embodiment, the DNA6 sequence comprises a double-stranded 5′ end (65) and/or a double-stranded 3′ end (63). In one embodiment, the DNA6 sequence comprises a portion base-paired (hybridized) to DNA5.

In one embodiment, the solution suitable for DNA ligation has the following components: DNA ligase, buffers, substrates, and other molecules appropriate to a DNA ligation reaction, and optionally Polynucleotide Kinase, and buffers, substrates, and other molecules appropriate to a Polynucleotide Kinase reaction

In one embodiment, the DNA is used to detect where a polypeptide or protein is bound along a DNA sequence. In one embodiment, the protein or polypeptide is crosslinked to DNA. In one embodiment, DNA cleavage activity is applied to the immobilized crosslinked material (e.g. U.S. Pat. No. 8,367,334 patent, “ChIP-exo 1.0”, i.e., Chromatin Immunoprecipitation or “ChIP”), and consisting of one of the following versioned series of steps:

Version 3.0

In one embodiment, the method comprises a step of reacting a modified low target specificity transposase (e.g., Tn5), bound to ligatable double-stranded DNA sequences (adapters), to the plurality of immobilized DNA.

In one embodiment, the method comprises one or more stringent wash steps that are sufficient to remove the plurality of bound Tn5.

In one embodiment, the method comprises DNA polymerization extending from the plurality target DNAs and through the attached adapters.

Version 4.0

In one embodiment, the method comprises a step of polishing or approximate blunt-ending of DNA molecules using one or more 5′-3′ DNA polymerases, and one or more strand-specific 3′-5′ exonuclease, to the plurality of immobilized DNA.

In one embodiment, the method comprises a step of directional and partial removal of one strand (e.g., 5′-3′) of double stranded DNA up to a fixed distance from the site of crosslinking, using a strand cleaving activity (e.g., Lambda exonuclease), to the plurality of immobilized DNA.

In one embodiment, the method comprises a step of conducting DNA ligation through a method including, but not limited to, ssDNA ligation.

In one embodiment, the method comprises a step of reversing the crosslink (e.g., with heat), and eluting the DNA from the immobilized resin (e.g., heat, detergent, and/or proteinase).

In one embodiment, the method comprises a step of conducting DNA ligation through a method including, but not limited to, splint ligation on purified eluted DNA.

Version 4.1

In one embodiment, the method comprises a step of polishing or approximate blunt-ending of DNA molecules using one or more 5′-3′ DNA polymerases, and one or more strand-specific 3′-5′ exonuclease, to the plurality of immobilized DNA.

In one embodiment, the method comprises a step of directional and partial removal of one strand (e.g., 5′-3′) of double stranded DNA up to a fixed distance from the site of crosslinking, using a strand cleaving activity (e.g., Lambda exonuclease), to the plurality of immobilized DNA.

In one embodiment, the method comprises a step of conducting DNA ligation through a method including, but not limited to, splint ligation.

In one embodiment, the method comprises a step of reversing the crosslink (e.g., with heat), and eluting the DNA from the immobilized resin (e.g., heat, detergent, and/or proteinase).

In one embodiment, the method comprises a step of conducting DNA ligation through a method including, but not limited to, splint ligation on purified eluted DNA.

Version 5.0

In one embodiment, the method comprises a step of A-tailing the plurality of immobilized DNA molecules (e.g., Klenow).

In one embodiment, the method comprises a step of phosphorylating the 5′ ends the plurality of immobilized DNA molecules (e.g., T4 polynucleotide).

In one embodiment, the method comprises a step of conducting DNA ligation through a method including, but not limited to, splint ligation.

In one embodiment, the method comprises simultaneously or sequentially conducting the phosphorylation and ligation steps.

In one embodiment, the method comprises a step of applying a 5′-3′ DNA polymerases to the plurality of immobilized DNA molecules (e.g. phi-29 DNA polymerase).

In one embodiment, the method comprises a step of directional and partial removal of one strand (e.g., 5′-3′) of double stranded DNA up to a fixed distance from the site of crosslinking, using a strand cleaving activity (e.g., Lambda exonuclease), to the plurality of immobilized DNA.

In one embodiment, the method comprises a step of reversing the crosslink (e.g., with heat), and eluting the DNA from the immobilized resin (e.g., heat, detergent, and/or proteinase).

In one embodiment, the method comprises a step of conducting DNA ligation through a method including, but not limited to, splint ligation on purified eluted DNA.

In one embodiment, DNA cleavage activity is NOT applied to the immobilized crosslinked material (standard ChIP), and consisting of the following series of steps:

One-Step ChIP-Seq and MNase ChIP-Seq

In one embodiment, the method comprises a step of conducting DNA ligation through a method including, but not limited to, ssDNA ligation, on the plurality of immobilized DNA.

In one embodiment, the method comprises a step of conducting DNA ligation through a method including, but not limited to, splint ligation, on the plurality of immobilized DNA.

In one embodiment, the method comprises simultaneously or sequentially ligating two adaptor molecules to the target nucleic acid molecule.

In one embodiment, the method comprises a step of reversing the crosslink (e.g., with heat), and eluting the DNA from the immobilized resin (e.g., heat, detergent, and/or proteinase).

In one embodiment, the method comprises reversing the crosslink prior to ligation of two adaptor molecules to the target nucleic acid molecule

In one embodiment, the protein or polypeptide is NOT crosslinked to DNA

In one embodiment, the DNA cleavage activity is NOT applied to the immobilized material.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for identifying a binding site of a protein of interest, the method comprising the steps of:
a) immunoprecipitating a protein of interest bound to a nucleic acid molecule,
b) contacting the immunoprecipitated nucleic acid molecule with at least one 5′→3′ exonuclease to generate a single-stranded nucleic acid region on the nucleic acid molecule,
c) ligating a first adaptor molecule to the immunoprecipitated nucleic acid molecule while it remains immobilized,
d) eluting the nucleic acid molecule,
e) ligating a second adaptor molecule to the eluted nucleic acid molecule,
f) amplifying the eluted nucleic acid molecule, and
g) sequencing the amplified products.

2. The method of claim 1, wherein step c) comprises ligating the first adaptor molecule by a method selected from the group consisting of tagmentation, 5′ ssDNA ligation, 3′ ssDNA ligation, splint ligation of an adaptor molecule having a 5′ ssDNA overhang, and split ligation of an adaptor molecule having a 3′ ssDNA overhang.

3. The method of claim 2, wherein the adaptor molecule comprises at least one of a 5′ ssDNA overhang comprising at least 2 random nucleotides at the 5′ end of the 5′ overhang and a 3′ ssDNA overhang comprising at least 2 random nucleotides at the 3′ end of the 3′ overhang.

4. The method of claim 1, wherein step e) comprises ligating the second adaptor molecule by a method selected from the group consisting of tagmentation, 5′ ssDNA ligation, 3′ ssDNA ligation, splint ligation of an adaptor molecule having a 5′ ssDNA overhang, and split ligation of an adaptor molecule having a 3′ ssDNA overhang.

5. The method of claim 4, wherein the adaptor molecule comprises at least one of a 5′ ssDNA overhang comprising at least 2 random nucleotides at the 5′ end of the 5′ overhang and a 3′ ssDNA overhang comprising at least 2 random nucleotides at the 3′ end of the 3′ overhang.

6. The method of claim 1, wherein the nucleic acid molecule is crosslinked to the protein of interest, and wherein the method further comprises a step of reversing the crosslinks after ligation of a first adaptor molecule.

7. The method of claim 1, wherein the method further comprises a step of end repair prior to exonuclease digestion.

8. The method of claim 1, wherein step c) is performed prior to step b).

9. The method of claim 8, wherein the method further comprises at least one step selected from the group consisting of:
h) performing A-tailing prior to ligation of a first adaptor molecule, and
i) phosphorylating a 5′ end of a nucleic acid molecule.

10. The method of claim 9, wherein the method comprises step i) and further wherein step i) is performed concurrently with step c).

11. The method of claim 8, wherein the method further comprises contacting the nucleic acid molecule with a polymerase to generate a completely dsDNA molecule by filling any ssDNA gaps in the nucleic acid molecule prior to step b).

12. A method for identifying a binding site of a protein of interest, the method comprising the steps of:
a) immunoprecipitating a protein of interest bound to a nucleic acid molecule,
b) ligating a first adaptor molecule to the immunoprecipitated nucleic acid molecule,
c) ligating a second adaptor molecule to the immunoprecipitated nucleic acid molecule,
d) eluting the nucleic acid molecule,
e) amplifying the eluted nucleic acid molecule, and
f) sequencing the amplified products.

13. The method of claim 12, wherein step b) is performed concurrently with step c).

14. The method of claim 12, wherein step b) comprises ligating the first adaptor molecule by a method selected from the group consisting of tagmentation, 5′ ssDNA ligation, 3′ ssDNA ligation, splint ligation of an adaptor molecule having a 5′ ssDNA overhang, and split ligation of an adaptor molecule having a 3′ ssDNA overhang.

15. The method of claim 14, wherein the adaptor molecule comprises at least one of a 5′ ssDNA overhang comprising at least 2 random nucleotides at the 5′ end of the 5′ overhang and a 3′ ssDNA overhang comprising at least 2 random nucleotides at the 3′ end of the 3′ overhang.

16. The method of claim 12, wherein step c) comprises ligating the second adaptor molecule by a method selected from the group consisting of tagmentation, 5′ ssDNA ligation, 3′ ssDNA ligation, splint ligation of an adaptor molecule having a 5′ ssDNA overhang, and split ligation of an adaptor molecule having a 3′ ssDNA overhang.

17. The method of claim 16, wherein the adaptor molecule comprises at least one of a 5′ ssDNA overhang comprising at least 2 random nucleotides at the 5′ end of the 5′ overhang and a 3′ ssDNA overhang comprising at least 2 random nucleotides at the 3′ end of the 3′ overhang.

18. The method of claim 12, wherein the nucleic acid molecule is crosslinked to the protein of interest, and wherein the method further comprises a step of reversing the crosslinks after ligation of a first adaptor molecule.

19. A method for identifying a binding site of a protein of interest, the method comprising the steps of:
a) immunoprecipitating a protein of interest bound to a nucleic acid molecule,
b) contacting the immunoprecipitated nucleic acid molecule with at least one transposase bound to an adaptor molecule,
c) washing the immunoprecipitated nucleic acid molecule at least once with a chaotrophic wash buffer,
d) contacting the immunoprecipitated nucleic acid molecule with least one 5′→3′ exonuclease to generate a single-stranded nucleic acid region on the nucleic acid molecule,
e) eluting the nucleic acid molecule,
f) contacting the eluted nucleic acid molecule with a non-specific primer and a polymerase for primer extension to generate a dsDNA molecule,
g) performing A-tailing on the eluted nucleic acid molecule,
h) ligating a second adaptor molecule to the eluted nucleic acid molecule,
i) amplifying the eluted nucleic acid molecule, and
j) sequencing the amplified products.

20. The method of claim 19, wherein the transposase is a hyperactive Tn5 with reduced target sequence specificity.
